(19) United States
(12) Patent Application Publication
(10) Pub. No.: US 2006/0019410 A1
(43) Pub. Date: Jan. 26, 2006

(54) Apparatus, kits and methods for evaluating binding interactions, for detecting and quantifying binding molecules, and for sample preparation

(75) Inventor: Robert Lee St. Claire III, Chapel Hill, NC (US)

Correspondence Address: MYERS BIGEL SIBLEY & SAJOVEC, PO BOX 37428, RALEIGH, NC, 27627, US

(73) Assignee: Qualyst, Inc., Research Triangle Park, NC (US)

(21) Appl. No.: 11/186,272

(22) Filed: Jul. 21, 2005

Related U.S. Application Data
(60) Provisional application No. 60/589,697, filed on Jul. 21, 2004.
(60) Provisional application No. 60/668,723, filed on Apr. 6, 2005.

(51) Int. Cl. C12M 1/22; G01N 33/537

(52) U.S. Cl. 436/538; 435/305.2

(57) ABSTRACT

The present invention provides apparatus, kits and methods for evaluating binding between one or more binding molecules (e.g., a protein) and one or more ligands. The invention also provides apparatus, kits and methods for detecting and/or quantifying a binding molecule, for example, a protein. Also provided are apparatus, kits and methods for “stripping” a complex biological matrix of low molecular weight components. The invention can be carried out on a smaller process scale, and therefore be more efficient, than previously known methods. The present invention is particularly suitable for use in high-throughput assays, which can be partially or completely automated.

### RELATED APPLICATION INFORMATION

This application claims the benefit of U.S. provisional application Ser. No. 60/589,697, filed 21 Jul. 2004, and U.S. provisional application Ser. No. 60/668,723 filed 6 Apr. 2005, the disclosures of which are incorporated herein by reference in their entireties.

### FIELD OF THE INVENTION

The present invention concerns apparatus for evaluating binding between a binding molecule and a ligand, for detecting and/or quantifying binding molecules, and for sample preparation, as well as methods for evaluating binding between a binding molecule and a ligand, method of detecting and/or quantifying binding molecules, and methods for sample preparation.

### BACKGROUND OF THE INVENTION

In vitro techniques for the analysis of ligand affinity and the extent of protein binding include equilibrium dialysis, ultrafiltration and ultracentrifugation. In the case of equilibrium dialysis and ultrafiltration, the protein of interest and a ligand are allowed to reach equilibrium binding in the presence of a semi-permeable membrane that permits movement of unbound ligand and restricts movement of bound ligand (Pacifici G M & Viani A, (1992) Clin. Pharmacokinet. 23:449-468). In the case of ultracentrifugation, protein-bound ligand is separated from unbound ligand by forcing the protein out of solution. However, non-specific binding of ligands to the membrane or to the apparatus can invalidate measurement of the unbound fraction. For some ligands, the extent of binding to a target protein cannot be reliably analyzed using available methods. Further, conventional membrane-based methods are labor-intensive and slow, and therefore not amenable to high throughput analysis.

Determination of unbound ligand fraction is particularly relevant to drug biodistribution. In the case of intravenous administration of a drug compound, binding of the drug to plasma proteins can substantially limit delivery of the drug to the site in need of treatment. A determination of the degree of ligand binding to plasma proteins can be used to predict the disposition of the drug in the body (see, e.g., Parikh H H et al., (2000) Pharm Res 17:632-637; Trung A H et al., (1984) Biopharm Drug Dispos 5:281-290; Suarez Varela et al., (1992) J Pharm Sci 81:842-844; Ascoli G et al., (1995) J Pharm Sci 84:737-741; Barr J et al., (1985) Clin Chem 31:60-64; Mendel C M, (1990) J Steroid Biochem Mol Biol 37:251-255; and Mendel C M et al., (1990) J Steroid Biochem Mol Biol 37:245-250).

There exists a need in the art for rapid, high throughput methods for assessing protein-ligand binding and for detecting and measuring binding proteins.

### SUMMARY OF THE INVENTION

As one aspect, the present invention provides an apparatus comprising a multiwell plate, each well of the multiwell plate comprising:

(a) a bottom portion having an opening formed therein; and

(b) a packed dextran-coated activated charcoal (DCC) bed.

Optionally, the apparatus further comprises:

(c) a filter membrane on top of the packed DCC bed which covers the exposed upper surface thereof; and/or

(d) a filter membrane below the packed dextran-coated DCC bed, wherein the filter membrane covers the opening.

Also provided are kits comprising the apparatus of the invention packaged together with written instructions for methods for determining protein-ligand binding, protein detection and/or quantitation, sample preparation and/or diagnostic methods, and optionally additional reagents or apparatus for carrying out such methods.

As a further aspect, the invention provides a method of evaluating binding of a ligand to a target protein, wherein the method comprises:

(a) providing a sample comprising a target protein and a ligand, wherein the target protein and the ligand are suspected to form a reversibly bound complex;

(b) applying the sample to the packed DCC bed of an apparatus of the invention for a time sufficient for adsorption of unbound ligand to the DCC;

(c) eluting the sample from the packed DCC bed;

(d) filtering the eluted sample through the filter membrane below the packed DCC bed; and

(e) determining an amount of ligand in the eluted sample to thereby evaluate binding of the ligand to the target protein.

In representative embodiments, the method of evaluating binding of a ligand to a target protein is a competitive binding method comprising:

(a) providing a sample comprising a target protein and a first ligand, wherein the first ligand forms a reversible complex with the target protein;

(b) contacting the sample with a candidate second ligand for a time sufficient for displacement of the first ligand from the complex by the candidate second ligand;

(c) contacting the sample of (b) with the packed DCC bed of an apparatus of the invention for a time sufficient for adsorption of unbound first ligand to the DCC;

(d) eluting the sample from the packed DCC bed;

(e) filtering the eluted sample through the filter membrane below the packed DCC bed; and

(f) determining an amount of first ligand in the eluted sample to thereby evaluate binding of the candidate second ligand to the target protein.

As still a further aspect, the invention provides a method for evaluating the susceptibility of a candidate drug to binding a target protein, wherein the method comprises:

(a) providing a sample comprising a target protein and a ligand, wherein the ligand forms a reversible complex with the target protein;

(b) contacting the sample with a candidate drug for a time sufficient for displacement of the ligand from the complex by the candidate drug;

(c) applying the sample of (b) to the packed DCC bed of an apparatus of the invention for a time sufficient for adsorption of unbound ligand to the packed DCC bed;

(d) eluting the sample from the packed DCC bed;

(e) filtering the eluted sample through the filter membrane below the packed DCC bed; and

(f) determining an amount of ligand in the eluted sample to thereby evaluate the susceptibility of the candidate drug to binding the target protein.

As yet a further aspect, the invention provides a method for evaluating drug-drug interactions, wherein the method comprises:

(a) providing a sample comprising a target protein and a ligand; wherein the ligand forms a reversible complex with the target protein;

(b) contacting the sample with a first candidate drug in the presence of a second candidate drug for a time sufficient for displacement of the ligand from the complex by the first candidate drug;

(c) applying the sample of (b) to the packed DCC bed of an apparatus of the invention for a time sufficient for adsorption of unbound ligand to the packed DCC bed;

(d) eluting the sample from the packed DCC bed;

(e) filtering the eluted sample through the filter membrane below the packed DCC bed;

(f) repeating steps (a) to (e) in the absence of the candidate second drug; and

(g) determining an amount of ligand in the eluted sample in the presence of the second candidate drug and comparing with an amount of ligand in the absence of the candidate drug to thereby evaluate interactions between the first and second candidate drugs.

The invention also provides a method of measuring a target protein in a sample, wherein the method comprises:

(a) providing a sample comprising a ligand, wherein the sample is suspected of comprising a target protein that forms a reversible complex with the ligand;

(b) applying the sample to the packed DCC bed of an apparatus of the invention for a time sufficient for adsorption of unbound target ligand to the DCC;

(c) eluting the sample from the packed DCC bed;

(d) filtering the eluted sample through the filter membrane below the packed DCC bed; and

(e) determining an amount of ligand in the eluted sample to thereby measure the target protein in the sample.

In addition, the invention provides a method of detecting the presence or absence of a target protein in a sample, wherein the method comprises:

(a) providing a sample comprising a ligand, wherein the sample is suspected of comprising a target protein that forms a reversible complex with the ligand;

(b) applying the sample to the packed DCC bed of an apparatus of the invention for a time sufficient for adsorption of unbound ligand to the DCC;

(c) eluting the sample from the packed DCC bed;

(d) filtering the eluted sample through the filter membrane below the packed DCC bed; and

(e) determining the presence of the ligand in the eluted sample, wherein the presence of the ligand in the eluted sample indicates that a target protein that binds to the ligand is present in the sample.

As another aspect, the invention provides a method for preparing a sample by reducing an amount of low molecular weight components in the sample, wherein the method comprises:

(a) providing a sample comprising a biological matrix;

(b) applying the sample to the packed DCC bed of an apparatus of the invention for a time sufficient for adsorption of low molecular weight components to the packed DCC bed; and

(c) eluting the sample from the packed DCC bed to thereby prepare a sample having a reduced amount of low molecular weight components.

As a further aspect, the invention provides a method for preparing a sample by reducing an amount of low molecular weight components in the sample, wherein the method comprises:

(a) providing a sample comprising a biological matrix;

(b) applying the sample to the packed DCC bed of an apparatus of the invention for a time sufficient for adsorption of low molecular weight components to the packed DCC bed;

(c) eluting the sample from the packed DCC bed; and

(d) filtering the eluted sample through the filter membrane below the packed DCC bed to thereby prepare a sample having a reduced amount of low molecular weight components.

These and other aspects of the invention are set forth in more detail in the description of the invention that follows.

### BRIEF DESCRIPTION OF THE DRAWINGS

FIG. 1A shows a top view of one representative multiwell plate comprising a packed DCC bed of the invention.

FIG. 1B and FIG. 1C are enlarged side views of alternative embodiments of the invention.

FIGS. 2A, 2B and 2C are side views of an alternative embodiment of the invention.

FIGS. 3A and 3B are side views of one representative apparatus of the invention comprising three multiwell plates.

FIGS. 4A and 4B are side views of an alternative embodiment of the invention.

FIG. 5 shows the result of the study described in Example 1. Recovery of various drugs that bind to HSA (x-axis) as compared with “HSA unbound fraction” as described in International Patent Publication WO 03/015871. DZP=diazepam; VPA=valproic acid; DPH=5,5-diphenylhydantoin; VER=verapamil; PRO=propranolol.

FIG. 6 shows percent recovery of ibuprofen and four analogs versus DCC bed size from HSA. Percent recovery values are an average value (n=3); error bars represent standard deviation.

FIG. 7 shows percent recovery of ibuprofen and four analogs versus DCC bed size from human plasma. Percent recovery values are an average value (n=3); error bars represent standard deviation.

FIG. 8 shows the effect of drug concentration on percent recovery of drugs known to bind HSA. Percent recovery values are an average value (n=3); error bars represent standard deviation.

FIG. 9 shows the results of a dansylsarcosine displacement assay to measure ligand binding to HSA.

### DETAILED DESCRIPTION OF THE INVENTION

The present invention provides apparatus, kits and methods for evaluating binding between one or more binding molecules (e.g., a protein) and one or more ligands. The invention also provides apparatus, kits and methods for detecting and/or quantifying a binding molecule, for example, a protein. Also provided are apparatus, kits and methods for “stripping” a complex biological matrix of low molecular weight components. The invention can be carried out on a smaller process scale, and therefore be more efficient, than previously known methods. The present invention is particularly suitable for use in high-throughput assays, which can be partially or completely automated.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein or in attachments hereto are incorporated by reference in their entirety.

I. Definitions.

The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise.

“Binding molecule” as used herein refers to any molecule to which a ligand can bind and includes biopolymers. Suitable binding molecules include but are not limited to proteins, hormones, chromatin, carbohydrates, and nucleic acids. Suitable proteins include but are not limited to circulating proteins found in warm-blooded vertebrates (e.g., mammals including humans and avians), plasma proteins, receptors, binding proteins, and enzymes. In particular embodiments, the binding molecule has a molecular weight of greater than about 10,000 daltons.

The term “adsorption” as used herein refers to adherence of a molecule, including a protein or a ligand, to a surface (e.g., the surface of dextran-coated activated charcoal). Adsorption can occur at arbitrary sites on the surface.

Adsorption of a molecule to dextran-coated activated charcoal can be influenced by temperature, nature of a solvent comprising the molecule, charcoal surface area, pore structure, nature of the solute, pH, the presence of inorganic salts, and the availability of competing ligands. Most of the factors remain consistent when using a variety of adsorbing molecules, although some differences are in the nature of the molecule itself. Although considered a neutral substance, the net charge of the activated charcoal surface is negative due to surface adsorption of OH⁻ ions. In general, the lower the aqueous solubility and the larger the molecule (in a series of compounds of similar structure), charcoal adsorption is greater. For compounds with dissimilar structure, side groups, substituent position, and molecular structure can be important for dictating the extent of adsorption. Hydroxyl, amino and sulfonic groups usually decrease adsorption while nitro groups often increase adsorption. Aromatic compounds are more adsorbable than aliphatic compounds and branched-chain molecules are more adsorbable than straight-chain molecules. Thus, for performance of the inventive methods, the above-mentioned parameters for influencing adsorption of a molecule to activated charcoal can be modified to promote a level of adsorption suitable for carrying out the methods of the invention.

The term “similar,” as used herein to describe a protein that is “similar” to a target protein, refers to a protein suspected of having similar ligand-binding features. As a nonlimiting example, a protein derived from an alternative species that is homologous to a target protein can be described as “similar” to the target protein. To illustrate, bovine serum albumin is considered to be “similar” to human serum albumin or other plasma proteins having similar ligand-binding features.

The term “eluting” as used herein refers to separation of a sample from the packed dextran-coated activated charcoal (DCC) bed. In particular embodiments, eluting the sample, filtering the eluted sample and/or collecting the sample for analysis are performed simultaneously. Vacuum suction or centrifugal force can be applied to facilitate elution from the DCC bed.

As used herein, the term “binding” refers to site-specific, saturable, binding of a ligand to a target protein, which may be reversible or irreversible.

The term “equilibrium binding” refers to a situation wherein a rate of association of a ligand and a protein to form a complex is equal and opposite to a rate of dissociation of the complex.

II. Apparatus.

As one aspect, the present invention provides an apparatus comprising a multi-chambered device that can hold multiple (e.g., two or more) samples at a time. In particular embodiments, the apparatus comprises a multiwell plate, e.g., a plate with six or more wells, such as a 96-well, 384-well or 1536-well plate. The apparatus can be used to practice the methods of the invention.

The multiwell plate can be formed from any suitable material, including but not limited to silicon, silica, quartz, glass, controlled pore glass, carbon, alumina, titania, tantalum oxide, germanium, silicon nitride, zeolites, and gallium arsenide. Metals (e.g., gold, platinum, aluminum, copper, titanium, and their alloys), ceramics and polymers may also be suitable. Illustrative polymers include, but are not limited to, the following: polystyrene; poly(tetra)fluoroethylene (PTFE); polyvinylidenedifluoride; polycarbonate; polymethylmethacrylate; polyvinylethylene; polyethyleneimine; poly(etherether)ketone; polyoxymethylene (POM); polyvinylphenol; polylactides; polymethacrylimide (PMI); polyalkenesulfone (PAS); polypropylene; polyethylene; polyhydroxyethylmethacrylate (HEMA); polydimethylsiloxane; polyacrylamide; polyimide; and block-copolymers. The multiwell plate can also comprise a combination of any of the aforementioned materials.

In particular embodiments, the multiwell plate is formed from nonreactive plastic such as polypropylene or polyethylene. Such plates are readily available from a number of commercial sources.

The multiwell plate further comprises a packed dextran-coated activated charcoal (DCC) bed. DCC is well known in the art and is readily available from commercial sources (e.g., Sigma). DCC is as described in PCT Publication WO 03/015871 (incorporated by reference herein in its entirety). For example, in particular embodiments, the DCC comprises dextrans having an average molecular weight of about 35 kDa to about 200 kDa, from about 50 kDa to about 150 kDa, or from about 75 kDa to about 80 kDa. Further, in representative embodiments, the DCC comprises a fractional weight of about 10% to about 80% dextran, about 10% to about 50% dextran, or about 10% to about 20% dextran.

In exemplary embodiments, the average molecular weight and/or percentage of dextran coating is selected so as to minimally adsorb a target protein or other binding molecule of interest. Such selection can be made taking into consideration factors known to those skilled in the art such as shape, conformation and charge of the protein (or binding molecule). In the context of the present invention, minimal adsorption of a target protein (or other binding molecule) is adsorption of less than about 10%, 5%, 2% or less of the target protein. In particular embodiments, the DCC column (optionally, a preconditioned column) does not adsorb compounds with a molecular weight of greater than about 10,000 daltons.

The packed DCC bed can be formed in the well by any method known in the art. For example, a slurry can be added to the well and a vacuum or centrifugation employed to pack the bed or sputtering techniques can be used to deposit the DCC bed.

One exemplary apparatus comprises a multiwell plate, each well comprising: (a) a bottom portion having an opening formed therein; and (b) a packed DCC bed. The opening can consist of part or all of the bottom portion of the well.

Any suitable amount of DCC can be used in forming the packed DCC bed, for example, about 0.1, 0.25, 0.5, 1, 2, 3, 4, 5, 7.5, 10, 12.5, 15, 20, 25, 30 or 40 or more mg DCC can be added per well. Suitable ranges include from about 0.25 to about 5, 7.5, 10, 15, 20 or 30, about 0.5 to 4, 5, 7.5, 10 or 20, and about 1 to 2.5, 3, 5, 7.5 or 10 mg DCC per well. In particular embodiments, the packed bed is formed of about 0.5 to 15 mg DCC per well, about 7.5 to 15 mg DCC per well, about 0.5 to 5 mg DCC per well, about 1 to 3 mg DCC per well, or about 2.5 mg DCC per well.

In other embodiments, the packed DCC bed is from about 0.25 to 5 mm, about 0.25 to 3 mm, or about 0.5 to 2.5, 3, 4 or 5 mm in thickness. In other embodiments, the packed DCC bed is about 2 mm in thickness.

Methods of designing bed thickness and flow rate to enhance recovery for a fixed volume of sample are known in the art. For example, the packed bed should typically provide sufficient adsorption capacity, but for use in binding studies it should generally not be so deep (with increased sample residence time) that recovery is unduly compromised. In some instances, however, it may be desirable to employ a longer residence time (e.g., a deeper bed), for example, to enhance resolution and differentiation of high binding ligands. Further, when the apparatus is used to “strip” a sample as a sample preparation protocol (see Section IV below), a greater bed depth and/or larger bed volume can enhance the stripping properties of the bed.

The multiwell plate can optionally comprise a filter on top of the packed DCC bed. By “on top of” or like terms as used in this context it is not necessary that the filter and packed DCC bed are in direct contact (e.g., there may be an intervening layer or element such as a prefilter) as long as the filter is before the packed DCC bed with respect to the flow of sample through the well. In some embodiments of the invention, however, the packed DCC bed and the filter are in direct contact. Typically, the upper surface of the packed DCC bed is covered by the filter to prevent direct sample contact with the packed DCC bed and to control adsorption of components in the sample to the bed. In particular embodiments, the multiwell plate comprises a filter on top of the packed DCC bed that covers the exposed upper surface thereof.

Optionally, the multiwell plate comprises a filter that forms all or part of the bottom of each well, i.e., covers the opening formed in the bottom portion of each well. Alternatively, the bottom of the well can have an opening formed therein and the apparatus is on top of a filter such that the sample flows through the packed DCC bed, the opening in the bottom of the well, and then contacts the filter. In certain embodiments where the apparatus is used for “stripping” low molecular weight components, the apparatus does not comprise a filter below the packed DCC bed. As a further alternative, a drain can be attached to the opening formed in the bottom of each well, and a filter can be within the drain or the drain can lead to a filter.

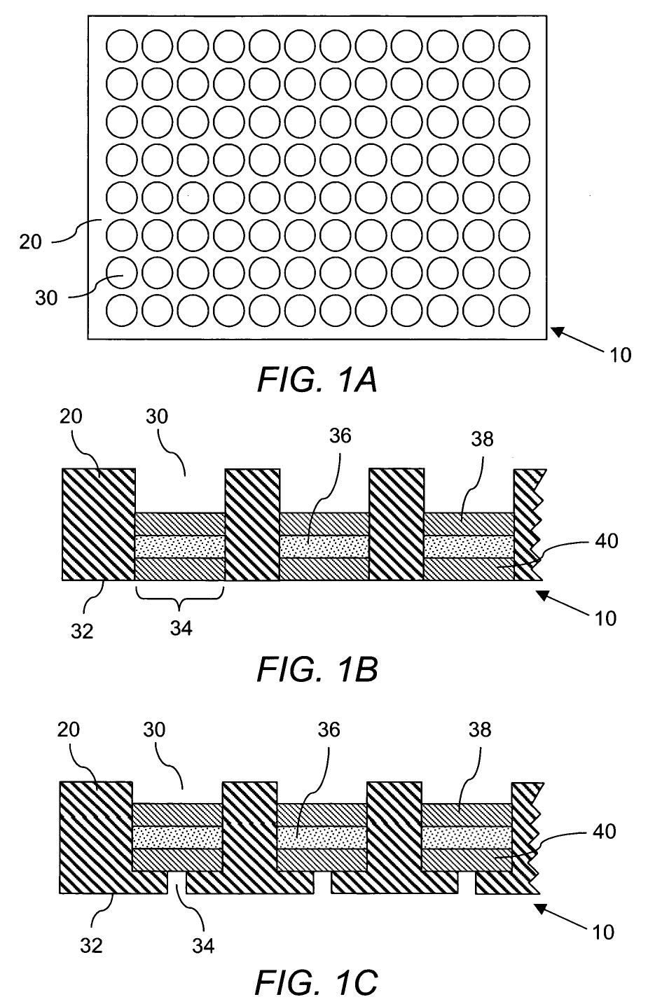

In particular embodiments, the apparatus comprises a filter below the packed DCC bed, wherein the filter covers the opening formed in the bottom portion of the well. The term “covers the opening” and like terms are intended to be broadly construed and include embodiments in which all of the portion of the well is open (i.e., the filter forms all of the bottom portion of the well; see, e.g., FIG. 1A), embodiments in which the opening forms part of the bottom portion of the well, and the filter is placed on either side of the remaining portions of the bottom portion of the well and the opening is formed (i.e., the filter forms part of the bottom portion of the well; see, e.g., FIG. 1B).

Further, the term “cover,” “covers” or like terms as used herein are not intended to require direct contact between the top filter and the exposed upper surface of the DCC bed or the bottom filter and the opening formed in the bottom portion of the well. For example, in particular embodiments, there can be an intervening layer or other element between the top filter and the DCC bed or the bottom filter and the opening formed in the bottom portion of the well (e.g., a support mat/drain as discussed herein). In other embodiments, there is direct contact between the top filter and the exposed upper surface of the DCC bed and/or there is direct contact between the bottom filter and the opening formed in the bottom portion of the well.

The packed DCC bed is generally on top of the filter in the multiwell plate. By “on top of” in this context it is not necessary that the filter and packed DCC bed are in direct contact (e.g., there may be an intervening layer or element such as a prefilter) as long as the packed DCC bed is before the filter with respect to the flow of sample through the well. Likewise, by saying that the filter is “below” the DCC bed, does not mean that the filter and packed DCC bed are necessarily in direct contact as long as the filter is after the packed DCC bed with respect to the flow of sample through the well. In particular embodiments, the DCC bed and filter are in direct contact.

In representative embodiments of the invention shown in FIG. 1A, the apparatus 10 comprises a multiwell plate 20 comprising a plurality of wells 30. Each well 30 of the multiwell plate 20 comprises: (a) a bottom portion 32 having an opening formed therein 34; (b) a packed DCC bed 36, (c) a filter membrane 38 on top of the packed DCC bed 36 and which covers the exposed surface thereof; and (d) a filter membrane 40 below the packed DCC bed 36, wherein the filter membrane 40 covers the opening 34 formed in the bottom portion 32 of well 30 (FIGS. 1B and 1C). FIG. 1B shows an embodiment in which the opening 34 encompasses all of bottom portion 32 of well 30 and is covered by filter membrane 40, i.e., filter membrane 40 forms the bottom portion 32 of well 30. FIG. 1C shows an alternate embodiment in which the opening 34 is a part of the bottom portion 32 of well 30 and is covered by filter membrane 40, i.e., filter membrane 40 forms part of bottom portion 32 of well 30.

Note that the bottom portion 32 in FIG. 1C is an extended bottom portion as compared with the bottom portion 32 in FIG. 1B to form a smaller opening 34 in the bottom portion 32 of multiwell plate 20 in FIG. 1C as compared with the opening 34 in the bottom portion 32 of multiwell plate 20 of FIG. 1B.

The filters can be any suitable filter known in the art, e.g., glass wool or a filter membrane. In general, the filter should be wettable with an aqueous solution so that aqueous samples can flow therethrough. Filters compatible with nonpolar samples are also known in the art. In addition, the filter should have low nonspecific binding properties (e.g., to the ligand). Suitable membranes include hydrophilic low protein binding membranes, such as a Hydrophil Durapore® membrane.

The top filter is generally selected so that the sample does not enter the packed DCC bed until application of vacuum or other force (e.g., by centrifugation). For example, a hydrophobic membrane can be used with an aqueous sample, and/or the pore size or surface tension can be selected so that the sample does not passively leak through the membrane (i.e., in the absence of external force) before it is desired to contact the packed DCC bed with the sample.

The bottom filter typically should not have so large a pore size that the charcoal can leak out of the well.

The pore size of the filter membranes can be chosen to achieve the desired flow rate and filtration properties. For example, the pore size should not be so small that the protein(s) and protein-ligand complex(es) of interest (or other binding molecules) are excluded and cannot pass through the membrane or that flow rate is so slow through the membrane and the DCC bed so as to have an undue adverse impact on recovery. In representative embodiments, the pore size is 0.65 or 1.2 μm.

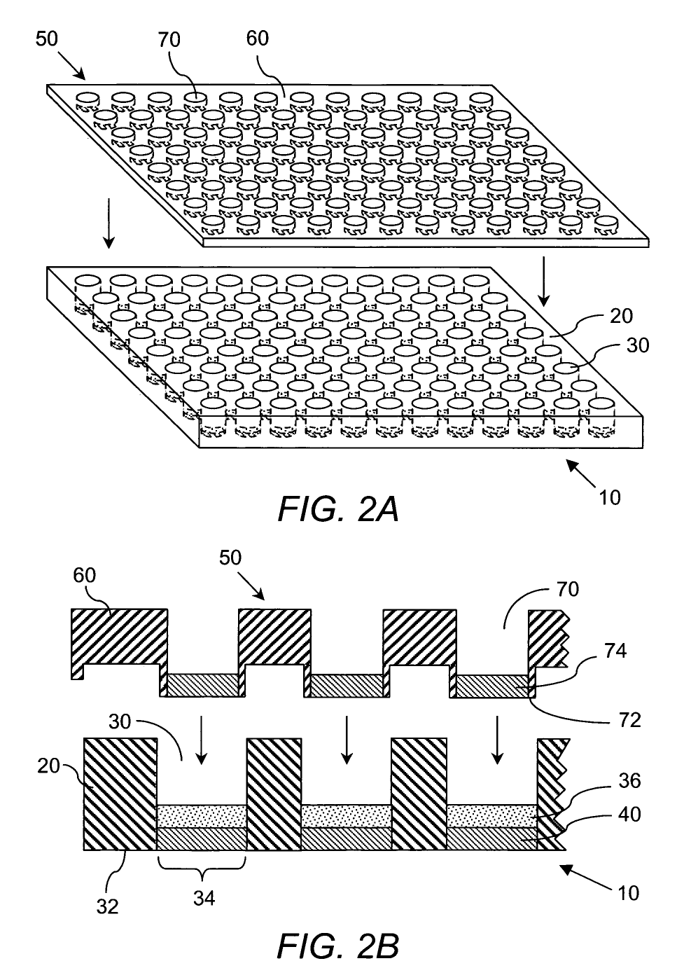

The filters can be held in place by any suitable method known in the art, e.g., by heat sealing, by an adhesive, or by an insertable device that is sealed into the well (see, e.g., FIGS. 2A, 2B and 2C).

In exemplary embodiments, the apparatus comprises a commercially available multiwell filtration plate such as the Millipore Multiscreen HTS™ DV filter plate comprising a Hydrophil Durapore® membrane (Millipore, Bedford, Mass.) with the addition of a DCC packed bed formed within each well, and optionally a second filter membrane on top of the bed (as described above; see, for example, FIG. 1B). 3M, Waters and Whatman also manufacture suitable hydrophilic low protein binding multiwell filtration plates.

In an alternative embodiment shown in FIG. 2A, the apparatus 10 further comprises a device 50 that fits over the multiwell plate 20 wherein the device comprises a plate 60 with projections 70 that fit into the individual wells 30 of the multiwell plate 20. The bottom portion 72 of each projection 70 comprises a filter 74 that covers the exposed upper surface of the packed DCC bed 36 when fitted into place over multiwell plate 20 (FIG. 2B). Optionally, the projections 70 further comprise a seal 80, such as an o-ring, that seals each projection 70 of device 50 into place in well 30 of multiwell plate 20 and prevents sample escaping from the top portion of well 30 (FIG. 2C).

The apparatus can optionally comprise one or more drains, which can lead from each well of the multiwell plate, through which the eluted sample can flow. Optionally, the drain(s) is connected to a vacuum device or another device that can push or pull a sample through the apparatus at an accelerated rate. Alternatively, centrifugation can be used.

Optionally, the apparatus comprises a support mat and drain (e.g., polypropylene) that supports filter membrane 40 and further acts as a drain. The support mat/drain is generally positioned to provide support to filter membrane 40 and can be directly below the filter (e.g., in the embodiment in FIG. 1C) or can be a mat that fits below the entire plate (e.g., in the embodiment in FIG. 1B). Such support mat and drain devices are incorporated into filtration plate available from Millipore.

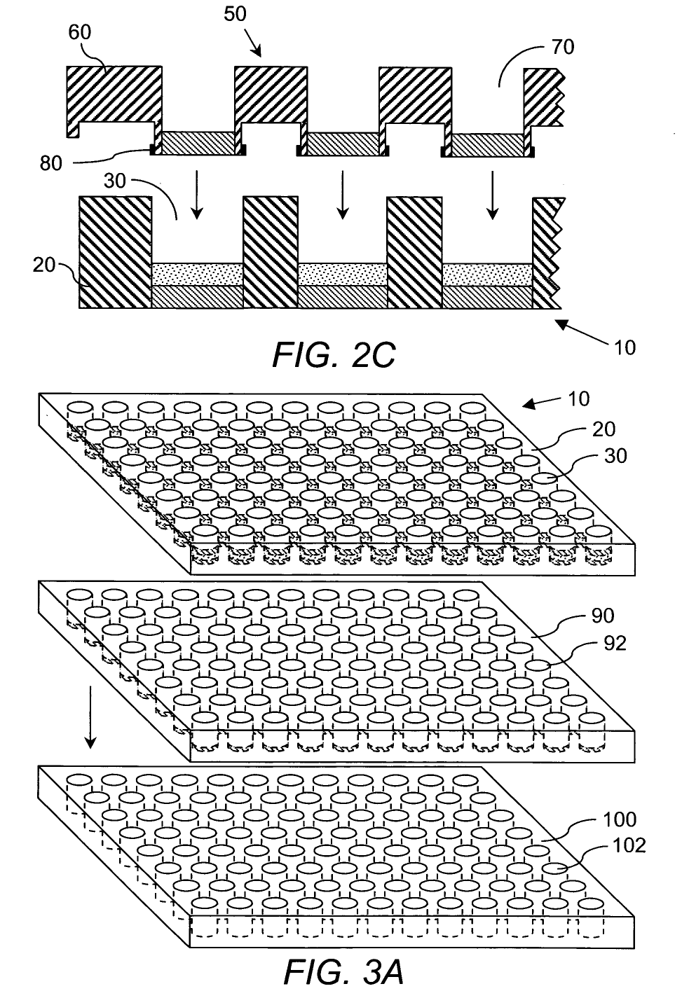

Additionally, as shown in FIGS. 3A and 3B, the eluted sample from multiwell plate 20 can be collected in a second multiwell filtration plate 90. When the second multiwell plate 90 is a filtration plate, the apparatus may further comprise a third multiwell plate 100 in series into which the filtrate from the second multiwell filtration plate 90 is collected. In general, the second multiwell filtration plate 90 comprises a plurality of wells 92, wherein each well 92 of the multiwell filtration plate 90 comprises a bottom portion 94 having an opening formed therein 96 and a filter membrane 98 which covers the opening 94 formed in the bottom portion 94 of well 92 (FIG. 3B). Filtrate from the second multiwell filtration plate 90 is collected in the corresponding plurality of wells 102 of the third multiwell plate 100.

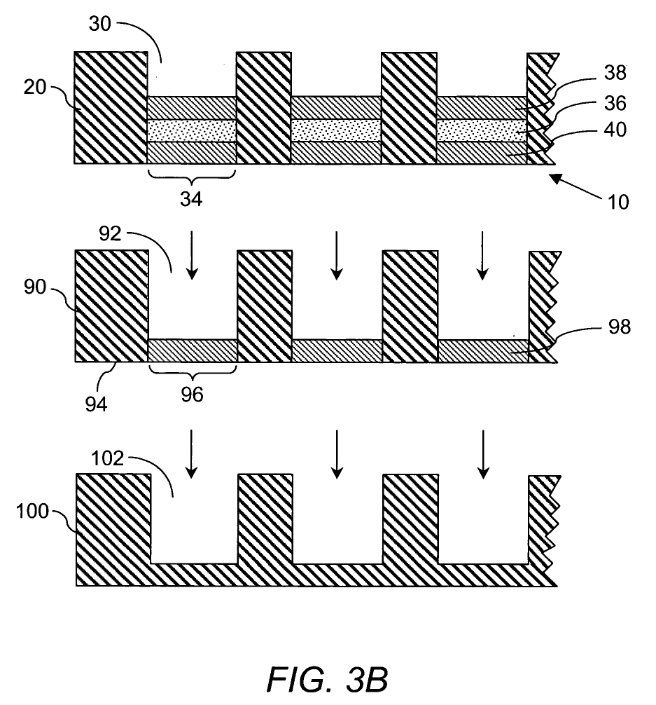

As another possible configuration, shown in FIGS. 4A and 4B, the filtrate from the first multiwell filtration plate 20 is collected into a multiwell transfer plate 110 (which is typically not a filtration plate) comprising a plurality of wells 112, and all or a portion of each sample is transferred to a second multiwell filtration plate 90, and the second filtrate from second multiwell filtration plate 90 collected into multiwell collection plate 100 as described above.

Any suitable filter (e.g., a filter membrane) can be used in the second multiwell filtration plate. In general, the filter will be selected to withstand relatively high concentrations of organic solvents and to have a small enough pore size to retain denatured proteins (e.g., 0.45 μm or less). In particular embodiments, the filter is a hydrophobic filter membrane. The filter should generally exhibit low nonspecific binding (e.g., to ligand). One illustrative filtration plate is the Millipore Multiscreen® Deep Well Solvinert Filter plate, which comprises a chemically-resistant hydrophobic polytetrafluoroethylene (PTFE) filter membrane (pore size of 0.45 μm) and a polypropylene prefilter. Whatman, 3M and Waters also make suitable multiwell filtration plates for this purpose.

The filter in the second multiwell filtration plate covers the opening in the bottom of the well. As described above with respect to the first multiwell filtration plate, the opening can consist of part or all of the bottom portion of the well. The filter can be held in place by any suitable method known in the art, e.g., by heat sealing, by an adhesive, or by an insertable device that is sealed into the well.

As another aspect, the invention provides a kit comprising one or more of the apparatus of the invention packaged together with written instructions for methods for determining protein-ligand binding (or ligand binding to other binding molecules), protein detection and/or quantitation (or detection and/or quantitation of other binding molecules), sample preparation and/or diagnostic methods, and optionally additional reagents or apparatus for carrying out such methods. In other embodiments, the invention provides a kit comprising a multiwell plate and DCC for preparing the apparatus of the invention packaged together with written instructions for methods for determining protein-ligand binding (or ligand binding to other binding molecules), protein detection and/or quantitation (or detection and/or quantitation of other binding molecules), sample preparation and/or diagnostic methods, and optionally additional reagents or apparatus for carrying out such.

III. Methods of Evaluating Binding of a Ligand to a Binding Molecule.

The apparatus and methods of the invention can be applied to a number of applications, including but not limited to methods for evaluating binding between a binding molecule(s) and ligand(s), methods of detecting and/or quantifying a binding molecule or ligand, methods of evaluating the susceptibility of a candidate drug to binding a binding molecule, methods of evaluating drug-drug interactions, and methods of identifying ligands for binding molecules.

The following discussion will focus on the practice of the invention to evaluate interactions between proteins and ligands and to detect and/or quantify proteins, but those skilled in the art will appreciate that these aspects of the invention are also applicable to other types of binding molecules.

A. Methods for Evaluating Binding of a Ligand(s) to a Target Protein(s).

According to this aspect, the apparatus of invention (described above in Section II) is employed in methods wherein the primary focus is on binding of one or more ligands to one or more target proteins of interest (e.g., a binding protein such as a receptor). The invention can be practiced to provide a quantitative assessment of the extent of ligand (e.g., drug) binding to a protein or proteins, optionally, within a biological matrix. According to this aspect of the invention, the identity, and optionally the amount, of the target protein(s) can be known. In other embodiments, e.g., evaluating protein-ligand binding in a complex matrix such as blood plasma, the identify of the protein(s) that is binding the ligand can be unknown. By “amount” of the target protein is generally known, it is not necessary that the absolute mass of the protein is known, but instead a relative amount of the protein can be known (e.g., in terms of volume of a biological sample, such as plasma). For example, a known amount of blood plasma can be added to the sample, even if it is not known how much of the protein is in the sample or even which protein(s) in the blood plasma is binding to the ligand. Optionally, the identity and/or amount of ligand in the sample is known as well.

As described in more detail below, the methods of the invention can be qualitative or quantitative in nature, and can be practiced in a “direct” (i.e., not based on competitive binding) or “competitive” binding format.

Accordingly, one embodiment of the invention comprises a method of evaluating binding of a ligand(s) to a target protein(s), wherein the method comprises: (a) providing a sample comprising a target protein and a ligand, wherein the target protein and ligand are suspected to be reversibly bound in a complex; (b) applying the sample to the packed DCC bed of the inventive apparatus for a time sufficient for adsorption of unbound ligand to the DCC; (c) eluting the sample from the DCC; (d) optionally filtering the eluted sample through the filter (if present) below the packed DCC bed; and (e) determining an amount of ligand in the eluted sample to thereby evaluate binding of the ligand to the target protein.

Packed DCC beds are as described above with respect to the inventive apparatus (Section II).

Without being limited by any theory of the invention, unbound target protein and target protein-ligand complexes will generally be too large to adsorb to the packed DCC bed, whereas unbound ligand will generally be retained by the bed. Thus, the amount of ligand detected in the eluate is directly related to the level of binding to the target protein. A highly bound ligand to the target protein will elute at a higher level (i.e., have a greater percent recovery from the bed) than a weakly bound ligand.

Parameters for designing packed DCC beds having specified levels of size exclusion to achieve the desired separation/retention of ligands and target proteins are discussed above and are also well-known in the art. Further, as also discussed herein and appreciated in the art, with larger bed volumes and/or increased bed depth and/or reduced flow rate (all which increase residence time), recovery can be diminished even for ligands that are highly bound to the target protein. Thus, bed volumes and dimensions can also be selected to yield the desired level of recovery and/or resolution between ligands (i.e., a larger bed volume and/or greater bed depth may exaggerate differences between ligands with relatively similar binding to the target protein, such as very highly bound ligands). Further, the DCC bed should generally be selected so that the target protein of interest is too large to be adsorbed by the bed, whereas unbound ligand will be adsorbed. To illustrate, as a non-limiting example, the DCC bed can be selected so that it adsorbs compounds that are less than about 10,000 daltons (e.g., ligands), but does not adsorb compounds that are greater than about 10,000 daltons (e.g., proteins). Those skilled in the art will appreciate that protein shape can influence whether the protein can pass through or is excluded from the dextran “net” on the charcoal.

Any suitable sample size can be applied to the packed DCC bed, for example, about 1 μl to about 250 or 500 μl, about 10 μl to about 150 or 200 μl, or about 20 μl to about 50 or 100 μl. In particular embodiments, about 50 μl of sample are added. The sample is generally aqueous when the binding molecule is a protein.

The methods of the invention can further comprise pre-incubating the target protein and ligand prior to contacting the sample with the packed DCC bed for a time sufficient for binding. For example, the target protein and ligand can be mixed together and incubated together before being applied to the packed bed. The term “time sufficient for binding” as used herein refers to a temporal duration that is sufficient for binding of a ligand to a target protein. The time sufficient for binding can be a time sufficient to achieve greater than about 50%, 75%, 90%, 95% or even 99% or greater equilibrium binding. In general, the “time sufficient for binding” can be any suitable time and in exemplary embodiments can be from about 0.5, 1, 2, 3, 4, 5, 10, 15, 20, 30 or 60 minutes until about 10, 15, 20, 30, 60, 90, 120, 150, 180, 240, 300, 480 or 600 minutes or longer at any suitable temperature (e.g., at room temperature, 37° C., or at 4° C.).

In practicing the invention, it may be advantageous to “precondition” the packed DCC bed so as to reduce adsorption of the target protein to the bed. Methods of preconditioning DCC are known by those skilled in the art. In particular embodiments, the DCC is pre-conditioned as described in PCT Publication WO 03/01581 with the target protein and/or with a protein similar to the target protein for a time sufficient for adsorption of the target protein and/or of the protein similar to the target protein to the DCC. Alternatively, any other protein that can reduce adsorption of the target protein to the packed DCC bed can be used (e.g., BSA or HSA). The DCC bed can be preconditioned for any suitable time, for example, at least about 1, 5, 10, 30, 60, 120 or 300 seconds.

The sample is contacted with (e.g., applied to) the packed DCC bed for a time sufficient for adsorption of unbound ligand to the packed DCC bed, which can be any suitable time. In general, however, one of the surprising advantages of the present invention is how rapidly it can be performed. The term “time sufficient for adsorption” as used herein refers to a temporal duration that is sufficient for adsorption of a ligand to the DCC. When a biological matrix comprising a ligand, a target protein, and complexes thereof, is applied to the DCC packed bed, preferably a “time sufficient for adsorption” does not disrupt equilibrium binding between the ligand and target protein. A time sufficient for adsorption can comprise a temporal interval to achieve adsorption of greater than about 50%, 75%, 90%, 95% or even 99% available unbound ligand. In particular embodiments, the time sufficient for adsorption of unbound ligand to the DCC bed is less than or equal to about 120, 90, 60, 45, 30, 20,15, 10, 8, 6, 5, 4, 3 or even 2 seconds.

In some embodiments, at least about 75%, 80%, 85%, 90%, 95% or even all of the free ligand is adsorbed.

Vacuum pressure or centrifugation can be used to increase the flow rate of the sample through the packed DCC bed. In some embodiments, the sample does not enter the bed prior to application of vacuum pressure or centrifugation.

The amount of the ligand in the eluted sample can be determined by any suitable method known in the art, including but not limited to mass spectrometry (e.g., LC/MS), immunoassay methods, gel electrophoresis, high performance liquid chromatography (HPLC), liquid scintillation counting, capillary electrophoresis, detection of a detectable label, and the like. See, e.g., Wahler D & Reymond J L (2001) Curr Opin Chem Biol 5:152-158; Maurer H H (2000) Comb Chem High Throughput Screen 3:467-480; and references cited therein.

In representative embodiments, the complex between the target protein and ligand is disassociated by denaturing the protein (e.g., chemical denaturation) following elution from the packed DCC bed and, if present, filtration through the bottom membrane, and the denatured target protein is optionally removed by centrifugation or further filtration to facilitate ligand detection. In methods wherein the ligand is detectably labeled, it generally is not necessary to dissociate the protein-ligand complex prior to detection.

In determining an “amount” of ligand, the amount can be a relative amount, a fractional amount, or an absolute amount.

In some instances, the determining step comprises determining the percent recovery of ligand through the apparatus, where percent recovery is equal to the amount of ligand that is recovered from the apparatus/method divided by the amount of ligand in a suitable control that has not been contacted with the packed DCC bed.

The invention is particularly suited for use with ligands that highly bind to the target protein, for example, highly plasma protein bound ligands. In particular embodiments, a ligand that highly binds to a target protein or to plasma proteins is at least about 95%, 97%, 99% or more protein or plasma protein bound, respectively. In evaluating binding of highly bound ligands, in some embodiments the residence time is increased (e.g., the bed volume and/or depth is increased and/or the flow rate through the column is reduced) to get improved resolution among ligands (i.e., to better distinguish the binding properties of ligands). For example, a bed volume of about 7.5 to 15 mg DCC (e.g., about 10 mg) can be used to form a packed bed in a well of a 96-well plate.

In particular embodiments, the methods of the invention are carried out on multiple ligands, either simultaneously in the same reaction, in parallel or sequentially, and the method further comprises ranking the ligands with respect to protein binding.

In representative embodiments of the invention, the ligand is detectably labeled and the method can further comprise detecting the detectably labeled ligand in the eluted sample.

Any suitable detectable label known in the art can by employed, although generally it is desirable that the label not unduly interfere with the protein-binding characteristics of the ligand. Suitable detectable labels include radiolabels, luminescent labels, epitope labels, colorimetric labels, or fluorescent labels. Generally, the detectable label should not be so large as to prevent adsorption/retention of the ligand by the packed DCC bed or interfere with protein binding.

In particular embodiments, the fluorescent label is a dansylamide or dansylsarcosine label, which bind to subdomains IIA and IIIA of human serum albumin, respectively, or a quinaldine red label, which is specific for the major binding site of AAG. Alternatively, the fluorescent label can be a fluorescent diazepam analog, which binds to subdomain IIA of HSA.

Methods for detectably labeling a ligand will vary depending on the molecular nature of the ligand. A typical method for detectably labeling a chemical compound is radiolabeling and can be accomplished using art-recognized techniques. Representative methods for protein labeling include but are not limited to radiolabeling, addition of biotin or any other epitope label by cross-linking or metabolic addition (Parrott M B & Barry M A, (2000) Mol. Ther. 1:96-104; Parrott M B & Barry M A, (2001) Biochem. Biophys. Res. Commun. 281:993-1000; and fluorescent labeling (Gruber H J et al., 2000). Techniques for labeling nucleic acid ligands include but are not limited to incorporation of labeled nucleotide analogues during nucleic acid replication, transcription, or amplification; addition of an end-label during a terminal transferase reaction; and formation of triplex structures. See, e.g., McPherson M et al. (eds.) (1995) PCR 2: A Practical Approach. IRL Press, New York; Sambrook J & Russell D (2001) Molecular Cloning: A Laboratory Manual. 3rd ed. Cold Spring Harbor Laboratory Press, Cold Spring Harbor, N.Y.; and Ausubel F (ed.) (1995) Short Protocols in Molecular Biology. 3rd ed. Wiley, N.Y.

Methods for detecting a labeled ligand are selected as appropriate for a type of label employed. For example, a radio-isotopic label can be detected using liquid scintillation spectrometry. A fluorescent label can be detected directly using emission and absorbance spectra that are appropriate for the particular label used. Fluorescent tags also include sulfonated cyanine dyes that can be detected using infrared imaging.

The term “mass spectrometry” as used herein refers to techniques including but not limited to gas chromatography-mass spectrometry (GC-MS), liquid chromatography-mass spectrometry (LC-MS), laser-desorption mass spectrometry (LD-MS), matrix-assisted laser desorption/ionization mass spectrometry (MALDI-MS), time-of-flight mass spectrometry (TOF-MS), electrospray ionization mass spectrometry (ESI-MS); tandem mass spectrometry, field release mass spectrometry, and combinations thereof. See e.g., Maurer H H (2000) Comb Chem High Throughput Screen 3:467-480; Karas M et al. (2000) Fresenius J Anal Chem 366:669-676; Kowalski P & Stoerker J (2000) Pharmacogenomics 1:359-366; Griffiths W J et al. (2001) Biochem J 355:545-561; U.S. Pat. Nos. 6,107,623; 6,104,028; 6,093,300; 6,057,543; 6,017,693; 6,002,127; 5,118,937; 5,952,654; and references cited therein. Such techniques are known to one of skill in the art and representative protocols for sample preparation can be found, for example, in Gilar M et al. (2001) J Chromatogr A 909:111-135, U.S. Pat. No. 5,545,895, and references cited therein.

To facilitate analysis of multiple ligands, a multiplexing approach can be used similarly to that described previously as a “cassette-accelerated rapid rat screen” (Korfmacher W A et al., (2001), Rapid Commun. Mass Spectrom. 15:335-340). Briefly, duplicate samples are prepared for analysis of a single ligand to a single target protein. Following analysis, samples are pooled such that each pooled sample comprises multiple (e.g., about six) individual samples, or other desired number of samples. Mass spectrometry is streamlined by analyzing the samples as cassettes of multiple samples.

For simultaneous analysis of binding of multiple candidate ligands to a single target protein, the providing of a sample can comprise contacting a target protein with a plurality of candidate ligands for a time sufficient for binding of the target protein to one or more of the plurality of candidate ligands. When evaluating ligand binding in a sample comprising a target protein and a plurality of ligands, an amount (optionally a fractional amount) of each ligand can be determined in the eluted sample by using liquid chromatography coupled to tandem mass spectrometry (Berman J et al., (1997), J. Med. Chem. 40:827-829; McLoughlin D A et al., (1997) J. Pharm. Biomed. Anal. 15:1893-1901; Olah T V et al., (1997) Rapid Commun. Mass Spectrom 11:17-23; Beaudry F et al., (1998) Rapid Commun Mass Spectrom 12:1216-1222; Frick L et al., (1998) Medicinal Chemistry Research 8:472-477), fast-atom bombardment mass spectrometry (Newton R P et al., (1997) Rapid Commun. Mass Spectrom. 11:1060-1066; Walton T J et al., (1998) Rapid Commun. Mass Spectrom. 12:449-455; White R & Manitpisitkul P, (2001), Drug Metabolism and Disposition 29:957-966), high performance liquid chromatography (U.S. Pat. No. 5,993,662), or by detecting differentially labeled ligand. In representative embodiments, a plurality of candidate ligands in a sample comprises less than or equal to about ten candidate ligands.

According to the methods of the invention, the target protein can be any protein (or portion thereof) of interest for evaluating protein-ligand interactions including but not limited to plasma proteins, receptors, binding proteins, and enzymes. In particular embodiments, the target protein is a protein found in the circulating blood of a warm-blooded vertebrate (e.g., mammals including humans and avians). For example, the target protein can be a plasma protein, such as serum albumin (e.g., HSA) or AAG. Other suitable target proteins include retinoid binding protein, or thyroxin binding protein. Mammals include but are not limited to humans, non-human primates, dogs, cats, pigs, goats, sheep, cattle, horses, mice, rats and rabbits. Avian subjects include but are not limited to chickens, turkeys, ducks, geese, quail, and birds kept as pets (e.g., parakeets, parrots, macaws, cockatoos, and the like).

The target protein can be isolated or can be present in a mixture of proteins. The target protein can further be a protein found within a biological matrix, such as blood or blood plasma (e.g., human plasma). According to this particular aspect of the invention, the sample can comprise a biological matrix (e.g, plasma such as human plasma) comprising a target protein, and the step of providing a sample can comprise contacting the sample comprising the biological matrix with at least one ligand for a time sufficient for binding of the at least one ligand by the target protein(s) in the biological matrix. Further, contacting a biological matrix comprising a target protein with at least one ligand can comprise creating a suspension of the biological matrix comprising the target protein and the at least one ligand. A time sufficient for binding will typically comprise a duration equal to or less than about 0.5, 1, 2, 3, 4, 5, 10, 15, 20, 30 or 60 minutes until about 10, 15, 20, 30, 60, 90, 120, 150, 180, 240, 300, 480 or 600 minutes or longer at any suitable temperature (e.g., at room temperature, 37° C., or at 4° C.). In some embodiments of the invention, the target protein is serum albumin (e.g., bovine serum albumin or human serum albumin), α-acid-glycoprotein (AAG), retinoid binding protein, or thyroxin binding protein.

As used herein, the term “biological matrix” comprises any heterogeneous mixture, suspension or solution comprising a target protein. In a preferred embodiment, a biological matrix comprises blood plasma, including blood serum, from a warm-blooded vertebrate (e.g., a mammal or a human).

According to representative embodiments, the invention provides a method of evaluating binding of a ligand(s) to a target protein(s) in blood plasma, wherein the method comprises: (a) providing a sample comprising blood plasma and a ligand, wherein a target protein(s) in the blood plasma and the ligand are suspected to be reversibly bound in a complex; (b) applying the sample to the packed DCC bed of an apparatus of the invention for a time sufficient for adsorption of unbound ligand to the DCC; (c) eluting the sample from the DCC; (d) optionally filtering the eluted sample through the filter (if present) below the packed DCC bed; and (e) determining an amount of ligand in the eluted sample to thereby evaluate binding of the ligand to the target protein(s) in blood plasma. According to this embodiment, the target protein(s) can be isolated proteins or of present in blood plasma. Further, according to this aspect of the invention, the identity of the target protein(s) may or may not be known.

The ligand is typically exogenously added to the sample and can be any molecule known in the art, including but not limited to a protein, a peptide, a saccharide, an oligonucleotide, a lipid, a small chemical molecule, or combinations thereof. Suitable chemical molecules encompass numerous chemical classes, although typically they are organic molecules, and are optionally small non-oligomeric organic compounds having a molecular weight of more than about 100 and less than about 2,500 daltons. Small non-oligomeric organic compounds include a wide variety of organic molecules, such as heterocyclics, aromatics, alicyclics, aliphatics and combinations thereof, comprising steroids, antibiotics, enzyme inhibitors, peptide hormones, alkaloids, opioids, terpenes, porphyrins, toxins, catalysts, as well as combinations thereof. Exemplary ligands include ibuprofen, ibuprofen analogs (e.g., naproxen, ketorolac, ketoprofen, etodolac), 5′ 5′ diphenylhydantoin, valproic acid, propranolol, verapamil, diazepam, chlorpromazine, warfarin, diazepam, furosemide, dicloxacillin, phenytoin, quinidine, lidocaine, digoxin, gentamicin, and atenolol. Further, the ligand can be a probe, such as a fluorescent probe, including but not limited to dansylamide, dansylsarcosine, quinaldine red, or a fluorescent diazepam analog. In other embodiments, the ligand is a molecule that is sufficiently small to pass through the dextran “net” over the charcoal and be adsorbed by the DCC (e.g., less than about 10,000 daltons).

In particular embodiments, the ligand is a xenobiotic, a new chemical entity, a candidate drug, or a drug, for example, a compound being evaluated for ADMET properties, e.g., for the purpose of developing a xenobiotic compound with therapeutic properties in humans or other mammals. As used herein, a “drug” is “a chemical agent that affects processes of living” (Goodman and Gilman The Pharmacological Basis of Therapeutics (9th ed. 1996)).

In particular embodiments of the foregoing methods, the amount of ligand and/or the identity of the ligand in the sample is known.

The methods of the invention can be carried out for two or more cycles, i.e., the sample can be repeatedly passed through the packed DCC bed to achieve the desired end-result.

In particular embodiments, once the sample has been eluted from the packed DCC bed, it passes through a filter and is collected in a second multiwell plate. Optionally, a drain connects the two plates. If the ligand is detectably labeled, the amount of ligand can be determined in the eluted sample. In some embodiments, the protein-ligand complex is disassociated (e.g., the protein is denatured) to facilitate ligand detection and the denatured protein removed by centrifugation or filtration, for example, the second multiwell plate can be a filtration plate. If the second multiwell plate is a filtration plate, the filter is generally chosen to retain denatured proteins but to allow unbound ligand to pass through (e.g., 0.45 μM or less). The denatured protein can therefore be separated from the ligand by filtration, and the filtrate is collected in a third multiwell collection plate, which is typically not a filtration plate. The amount of ligand can then be determined from the free ligand that is collected following filtration to remove denatured protein. Alternatively, all or a portion of the denatured sample is removed from the second multiwell plate (which is not a filtration plate) to a multiwell filtration plate, and the free ligand is separated from the target protein by filtration through the second multiwell filtration plate into another multiwell collection plate.

One exemplary method of determining protein-ligand interactions with an apparatus of the present invention is illustrated in FIGS. 3A and 3B. According to this method, a sample comprising a ligand and target protein is applied to a packed DCC bed 36 of multiwell plate 20, typically for a time sufficient for adsorption of unbound ligand to packed DCC bed 36. A filter 40 covers the opening 34 in the bottom of the well 30. Packed DCC bed 36 is on top of membrane 40. Multiwell plate 20 can be a Millipore Multiscreen HTS™ DV filter plate comprising a Hydrophil Durapore® membrane and further comprising a packed DCC bed. The apparatus 10 further comprises a filter 38 on top of packed DCC bed 36 and covering the exposed upper surface thereof as described herein, which is optionally a Hydrophil Durapore® filter. The sample generally does not pass through top membrane 38 and enter the packed DCC bed 36 prior to applying vacuum pressure or centrifugation. In particular embodiments, the DCC packed bed 36 is preconditioned as described herein.

The free ligand in the sample is adsorbed to the packed bed DCC 36, whereas ligand bound to target protein (target protein-ligand) is not and passes through the packed DCC bed 36 and flows out of multiwell plate 20 into multiwell filtration plate 90, such as a Millipore Multiscreen® Deep Well Solvinert Filter plate (e.g., through a drain). In methods wherein the protein-ligand complex is denatured in multiwell filtration plate 90, the bottom filter 98 in multiwell filtration plate 90 is generally chosen to withstand higher concentrations of organic solvents and to have a small enough pore size to retain denatured proteins (e.g., 0.45 μm or less). Whatman, 3M and Waters also make suitable multiwell filtration plates. Free ligand is then filtered from multiwell filtration plate 90 into multiwell collection plate 100, which can be a commercially-available 2.4 ml deep multiwell plate. Vacuum pressure or centrifugation can be used to increase flow rate through bottom filter 98 in multiwell filtration plate 90. The amount of ligand in multiwell collection plate 100 can be determined as described above or by any method known in the art.

In particular embodiments, multiwell plate 20, multiwell filtration plate 90 and/or multiwell collection plate 100 are in a stacked arrangement.

Alternatively, as shown in FIGS. 4A and 4B, the filtrate from multiwell plate 20 can be collected in multiwell transfer plate 110, and then all or a portion of the collected sample transferred to multiwell filtration plate 90. Protein denaturation can be carried out in multiwell transfer plate 110 prior to transfer to multiwell filtration plate 90 for filtration and separation.

In methods in which protein-ligand disassociation by protein denaturation is not carried out (e.g., when the ligand is detectably labeled), the sample can be collected in multiwell transfer plate 110 and then an amount of ligand determined (e.g., by detecting the detectably labeled ligand) without further filtration and separation with multiwell filtration plate 90.

The methods can also be carried out in a competitive binding format. The term “competitive binding” as used herein refers to displacement of a first ligand from binding to a target protein by a second ligand. In some instances, competitive binding refers to displacement of a first ligand from a binding site on a target protein by a candidate second ligand that specifically binds the same site. A candidate second ligand can be identified as binding a target protein, optionally at a particular site, by observing displacement of a first ligand suspected or known to bind that same protein or site.

In some embodiments, either one or both of the first ligand and the candidate second ligand are a peptide, an oligonucleotide, or a small chemical molecule. Further, either one or both of the first ligand and the candidate second ligand can be drugs. In other particular embodiments, the first ligand and/or the candidate second ligand are ibuprofen, ibuprofen analogs (e.g., naproxen, ketorolac, ketoprofen, etodolac), 5′ 5′ diphenylhydantoin, valproic acid, propranolol, verapamil, diazepam, and/or chlorpromazine, warfarin, diazepam, furosemide, dicloxacillin, phenytoin, quinidine, lidocaine, digoxin, gentamicin, and atenolol. In other embodiments, one of first and second ligands is a fluorescent probe (e.g., dansylamide, dansylsarcosine, quinaldine red, or a fluorescent diazepam analog). In general, both the first and second ligands are small enough to pass through the dextran “net” and adsorb to the DCC in their unbound state (e.g., less than about 10,000 daltons).

To illustrate, in representative embodiments, the invention provides a method of evaluating binding of a candidate ligand to a target protein, wherein the method comprises: (a) providing a sample comprising a target protein and a first ligand; wherein the first ligand forms a reversible complex with the target protein; (b) contacting the sample with a candidate second ligand for a time sufficient for displacement of the first ligand from the complex by the candidate second ligand; (c) contacting the sample of (b) with the packed DCC bed of an apparatus of the invention for a time sufficient for adsorption of unbound first ligand to the DCC bed; (d) optionally filtering the eluted sample through the filter (if present) below the packed DCC bed; (e) eluting the sample from the packed DCC bed; and (f) determining an amount of first ligand in the eluted sample to thereby evaluate binding of the candidate second ligand to the target protein.

As discussed in more detail above, the amount of the first ligand in the eluted sample can be determined by any method known in the art, including but not limited to mass spectrometry, immunoassay methods, gel electrophoresis, detection of a detectable label, and the like.

When the first ligand is detectably labeled, the method can further comprise detecting the detectably labeled first ligand in the eluted sample. Detectable labels are as described above.

A “time sufficient for displacement of the first ligand from the complex by the second ligand” can be any suitable period of time (e.g., a duration less than or equal to about 120, 90, 60, 45, 30, 20, 15, 10, 5, 3, 2 or 1 minute or even 30, 20, 15, 10, 5, 4, 3, 2 or 1 second).

In particular embodiments, the identity and/or the amount of first ligand and/or the candidate second ligand are known.

According to this “competitive” assay embodiment of the invention, the method can further comprise employing a first ligand that comprises a ligand that binds a specific binding site of a target protein. In this case, an amount of the first ligand in the eluted sample is determined to thereby evaluate binding of the candidate second ligand to the specific ligand-binding site of a target protein.

For example, in particular embodiments of the invention, the first ligand binds a plasma protein, e.g., a ligand that binds to serum albumin (such as HSA) or AAG. More particularly, in exemplary embodiments, the first ligand binds to a specific site on a plasma protein such as serum albumin or AAG.

The term “specific binding site” or “specific ligand-binding site” and like terms refer to a ligand-binding site on a target protein that shows selective binding, e.g., binds to a subset of ligands.

To illustrate, at least six classes of primary (high-specificity) binding sites have been identified on HSA, and a larger number of secondary (lower specificity) binding sites. The warfarin site (site I) primarily interacts with coumarins, salicylates, and pyrazolidines, and the indole site (site II) specifically binds benzodiazepines, arylpropionates, and L-tryptophan. Site III can be specifically bound by digitoxin. Thus, a site-specific HSA ligand can be used in accordance with the methods of the present invention to evaluate binding of a candidate ligand (e.g., drug) to a particular binding site on HSA.

In representative embodiments, the first ligand comprises a ligand that binds a specific binding site of HSA, e.g., a site I, site II, or site III of serum albumin (e.g., HSA). The first ligand can comprise a site I-binding ligand such as a coumarin and/or a pyrazolidine, and can more specifically be selected from the group consisting of valproate, diphenylhydantoin, salicylate, and combinations thereof. Alternatively, the first ligand can comprise a site II-binding ligand such as a benzodiazepine, an arylpropionate, and/or L-tryptophan. As a further alternative, the first ligand can comprise a site III-binding ligand such as digitoxin.

In some embodiments of the invention, the step of providing a target protein and a first ligand comprises contacting a biological matrix comprising the target protein with the first ligand for a sufficient time for binding between the target protein and the ligand. Thus, the invention also provides a method of evaluating binding of a candidate ligand to a target protein(s) in blood plasma, wherein the method comprises: (a) providing a sample comprising blood plasma and a first ligand; wherein the first ligand forms a reversible complex with a target protein(s) in blood plasma; (b) contacting the sample with a candidate second ligand for a time sufficient for displacement of the first ligand from the complex by the candidate second ligand; (c) contacting the sample of (b) with the packed DCC of an apparatus of the invention for a time sufficient for adsorption of unbound first ligand to the packed DCC bed; (d) optionally filtering the eluted sample through the filter (if present) below the packed DCC bed; (e) eluting the sample from the DCC; and (f) determining an amount of first ligand in the eluted sample to thereby evaluate binding of the candidate second ligand to the target protein.

One application of the competitive assay described above is to evaluate drug-protein (e.g., plasma protein) interaction. According to one representative embodiment, the invention provides a method for evaluating the susceptibility of a candidate drug to binding a target protein (e.g., a protein found in the circulating blood of a warm-blooded vertebrate), wherein the method comprises: (a) providing a sample comprising a target protein and a ligand; wherein the ligand forms a reversible complex with the target protein; (b) contacting the sample with a candidate drug for a time sufficient for displacement of the ligand from the complex by the candidate drug; (c) applying the sample of (b) to a packed DCC bed of an apparatus of the invention for a time sufficient for adsorption of unbound ligand to the DCC bed; (d) eluting the sample from the packed DCC bed; (e) optionally filtering the eluted sample through the filter (if present) below the packed DCC bed; and (f) determining an amount of ligand in the eluted sample to thereby evaluate the susceptibility of the candidate drug to binding the target protein. A “time sufficient for displacement of the first ligand from the complex by the candidate drug” can be any suitable period of time (e.g., a duration less than or equal to 120, 90, 60, 45, 30, 20, 15, 10, 5, 3, 2 or 1 minute or even 30, 20, 15, 10, 5, 4, 3, 2 or 1 second). The target protein(s) in the blood plasma can be known or unknown. Similar methods can also be carried out to determine the susceptibility of a xenobiotic, a new chemical entity, or a drug to binding a target protein.

This aspect of the invention is particularly useful to determine an amount of a drug (e.g., a fractional amount) that is bound by plasma protein(s), although it may not be known to which protein(s) in plasma the ligand is binding. According to this embodiment, the sample can comprise blood plasma or isolated plasma proteins.

In particular embodiments, the amount of ligand and/or drug in the sample is known.

As another application of the competitive assay format, the invention can be practiced to evaluate drug-drug interactions. In this case, binding of a first drug to a target protein can be assessed in the presence and absence of a second drug, which may or may not competitively bind to the same target protein. This analysis can provide information on potential interactions between co-administered drugs. According to one particular embodiment, the invention provides a method for evaluating drug-drug interactions, wherein the method comprises: (a) providing a sample comprising a target protein and a ligand; wherein the ligand forms a reversible complex with the target protein; (b) contacting the sample with a first candidate drug in the presence of a second candidate drug for a time sufficient for displacement of the ligand from the complex by the first candidate drug; (c) applying the sample of (b) to the packed DCC bed of an apparatus of the invention for a time sufficient for adsorption of unbound ligand to the DCC bed; (d) eluting the sample from the packed DCC bed; (e) optionally filtering the eluted sample through the filter (if present) below the packed DCC bed; (f) repeating steps (a) to (e) in the absence of the candidate second drug; and (g) determining an amount of ligand in the eluted sample in the presence of the candidate second drug and comparing with an amount of ligand in the absence of the candidate second drug to thereby evaluate interactions between the first and second candidate drugs. A “time sufficient for displacement of the ligand from the complex by the first candidate drug” can be any suitable period of time (e.g., a duration less than or equal to 120, 90, 60, 45, 30, 20, 15, 10, 5, 3, 2 or 1 minute or even 30, 20, 15, 10, 5, 4, 3, 2 or 1 second).

In particular embodiments, the amount of ligand, first candidate drug and/or second candidate drug is known.

As discussed at some length above, the amount of the ligand in the sample can be determined by any method known in the art, including but not limited to mass spectrometry, immunoassay methods, gel electrophoresis, detection of a detectable label, and the like.

In the case that the ligand is detectably labeled, the method can further comprise detecting the detectably labeled ligand in the eluted sample (e.g., prior to the determining step). Detectable labels are as discussed above.

One competitive binding method that evaluates binding to a specific binding site of a target protein uses a fluorescent displacement assay in which unlabeled or differently labeled ligand displaces a fluorescent probe from its cognate binding site on the target protein. Suitable fluorescent probes for use in these methods include but are not limited to dansylamide and dansylsarcosine probes, which bind to subdomains IIA and IIIA of human serum albumin, respectively, and quinaldine red probes, which are specific for the major binding site of AAG. These probes are available from Sigma (St. Louis, Mo. USA). Other suitable probes include fluorescent diazepam analogs, which specifically bind to subdomain IIA of HSA. Fluorescent displacement assays are particularly useful with highly bound ligands as described above (e.g., a ligand that highly binds to a target protein or to plasma proteins is at least about 95%, 97%, 99% or more protein or plasma protein bound, respectively). Free probe can be detected by any method known in the art, for example by mass spectrometry or by detection of fluorescence.

Other aspects of the competitive assay methods of the invention are essentially as described hereinabove with respect to the direct (non-competitive) assay format.

The foregoing methods have a number of applications and can be used, for example, for drug metabolism protein binding screening. As an illustration, the present invention has been used to evaluate drug-human serum albumin (HSA) binding over a broad range of HSA-based affinities for drugs with high overall plasma protein binding (e.g., at least about 70%, 75%, 80%, 85%, 90%, 95% or 99%). Furthermore, the apparatus has been used in a system with non-homogenous proteins (e.g., human blood plasma, or a mixture of blood plasma proteins like HSA and AAG), to measure the specificity of a ligand binding to a specific protein. For example, in a mixture of HSA and AAG, the invention was able to show that a drug with a high affinity for AAG was selectively bound to this receptor in the presence of high amounts of HSA.

The methods described herein can also be used to evaluate competitive binding of ligands, and their interaction, to a target protein.

Performance of the inventive methods can further be included in a drug development program for predicting drug disposition and activity (see, e.g., Huang & Oie, (1982) J. Pharmacol. Exp. Ther. 223: 469-471 and Qin et al., (1994) J. Pharmacol. Exp. Ther. 269:1176-1181).

B. Methods of Qualitative and Quantitative Assessment of Proteins.

The apparatus of the present invention can further be applied to evaluate protein-ligand binding to detect and/or quantify the amount of a protein(s) in a sample. For example, the present invention has been used to detect, proportionally, AAG at 1× and 0.5× its typical human physiological concentration while in the presence of a physiologically relevant amount of HSA. There is great interest in measuring AAG levels to (i) assess disease states in humans and animals, and (ii) assess potential impact of AAG on the circulating drug levels of high-AAG binders in certain disease states. The basis of this aspect of the invention is similar to the protein binding methods described in Section IIIA above in that the amount of ligand in the eluted sample is directly proportional to the amount of the target protein in the sample.

According to this embodiment of the invention, the amount of target protein(s) in the sample is generally unknown. The identity of the target protein(s) may be known or unknown. The ligand(s) is typically known and, optionally, the amount of ligand is known as well. In the practice of this aspect of the invention, it is often desirable to use a highly specific and highly bound ligand.

The method can be quantitative, semi-quantitative and/or qualitative. For example, qualitative methods can be used to detect the presence or absence of a protein(s) that binds to the ligand(s), where the identity of the protein(s) may or may not be known. Semi-quantitative methods can be used to determine a level of a target protein(s) above a threshold value. Quantitative methods can be used to determine a relative or absolute amount of a target protein(s) in the sample. In this case, the identity of the protein is generally known.

Quantitative methods can provide absolute or relative measures of the amount of protein in the sample, and can be based on any methodology known in the art of protein quantitation. If the binding relationship between a known ligand and known binding protein is also known, then it is possible to calculate an absolute amount protein based on the amount of ligand in the eluted sample using standard mathematical models. Alternatively, a standard binding curve can be used to determine the amount of protein in a sample. If the nature of the interaction between the binding protein and the ligand is not known and/or if the identity of the protein is not known, then the method will generally determine a relative amount of protein in the sample, for example, as compared with other samples or with a standard.

In semi-quantitative methods, a threshold or cutoff value can be determined by any means known in the art, and is optionally a predetermined value. In particular embodiments, the threshold value is predetermined in the sense that it is fixed, for example, based on previous determinations of the presence of known amounts of the protein and/or previous assays. Alternatively, the term “predetermined” value can also indicate that the method of arriving at the threshold is predetermined or fixed even if the particular value varies among assays for the same ligand or may even be determined for every assay run.

In qualitative methods, the presence or absence of a target protein in a sample is determined. To illustrate, if no protein is present, then all or essentially all of the ligand will be adsorbed and retained by the packed DCC bed and essentially no ligand will be in the eluted sample. Those skilled in the art will appreciate that a background level of ligand may nonetheless be present and detected in the eluted sample, which can be taken into account by using proper controls and other methods known in the art. For example, the protein may be deemed to be present in the sample if ligand is detected in the eluted sample at greater than two-fold above background levels or is detected at levels greater than any other suitable control value that may be selected.

Thus, in particular embodiments, the invention provides a method of measuring a target protein in a sample, wherein the method comprises: (a) providing a sample comprising a ligand, wherein the sample is suspected of comprising a target protein that forms a reversible complex with the ligand; (b) applying the sample to the DCC bed of the apparatus of the invention for a time sufficient for adsorption of unbound ligand to the DCC bed; (c) eluting the sample from the packed DCC bed; (d) optionally filtering the eluted sample through the filter (if present) below the packed DCC bed; and (e) determining the amount of ligand in the eluted sample to thereby measure the target protein in the sample.

In determining an “amount” of ligand, the amount can be a relative amount, a fractional amount, or an absolute amount.

The term “measuring a protein” encompasses any type of measurement of a protein including but not limited to: (i) qualitative methods to determine the presence or absence of the protein in the sample; (ii) quantitative methods to determine a relative or absolute amount of the protein in the sample; and (iii) semi-quantitative to determine the presence or absence of the target protein in the sample above a threshold amount.

This aspect of the invention can be used to detect the presence or absence of a known or unknown protein in a sample, to quantify the amount of a known protein in a sample, to identify a binding protein for a ligand of interest, or as a diagnostic method. For example, the method can be practiced to determine the presence or absence or level of a marker protein or any other protein associated with a disease state to diagnose and/or monitor the disease state in a subject. In particular embodiments, the marker protein is a circulating protein (e.g., found in blood or blood plasma). By “associated with a disease state,” the presence or absence or amount of the protein can be causative of the disease state or can be reflective of a disease state. As one illustration, AAG levels in blood plasma are indicative of certain disease states (e.g., inflammation). Other illustrative proteins associated with specific disorders, which can be detected and/or quantified according to the present invention to diagnose and/or monitor a disorder in a subject (e.g., mammals such as humans and avians) include but are not limited to: autoantibodies (e.g., autoimmune disorders); alpha-feto protein (e.g., Down's syndrome, open neural tube defects such as spina bifida and anencephaly, and trisomy 18); Prostate Specific Antigen and/or prostatic acid phosphatase (e.g., prostate cancer); antibodies against a pathogen (e.g., infection); cancer specific antigens (e.g., cancer); C-reactive protein (e.g., inflammation, tissue injury, neoplastic disease, cardiac disease); liver enzymes such alanine amino transferase and AST (e.g., indicative of liver injury, liver function and liver disease such as cirrhosis, hepatitis, infectious mononucleosis, and Reye syndrome); and proteins that are defective or at reduced levels in inborn errors of metabolism including but not limited to: α-L-iduronidase (e.g., Hurler Syndrome [MPS III], Scheie Syndrome [MPS IS] and Hurler-Scheie Syndrome[MPS IH/S]), iduronate sulfatase (Hunter Syndrome; MPS II), heparan N-sulfatase (Sanfilippo A Syndrome; MPS IIIA), α-N-acetylglucosaminidase (Sanfilippo B Syndrome; MPS IIIB), acetyl-CoA-glucosaminide acetyltransferase (Sanfilippo C Syndrome; MPS IIIC), N-acetylglucosamine-6-sulfatase (Sanfilippo D Syndrome; MPS IIID), galactosamine-6-sulfatase (Morquio A disease; MPS IVA), β-galactosidase (Morquio B disease; MPS IV B), arylsulfatase B (Maroteaux-Lamy disease; MPS VI), and β-glucuronidase (Sly Syndrome; MPS VII).

The methods can also be practiced to determine which protein within a pathway the ligand interacts. Further, the methods can be practiced to assess the kinetics or physics of binding between the target protein and ligand.

The invention can also be practiced to identify an unknown protein(s) that binds to a ligand of interest. For example, fractions of blood plasma (e.g., human blood plasma) or any other protein mixture or biological matrix can be screened to identify the presence of a protein(s) that binds to the ligand of interest, although the identity of the binding protein(s) may not be known. According to this aspect, the invention can be used to identify binding proteins for ligands having an unknown binding partner. Thus, the invention also provides a method of detecting the presence or absence of a target protein in a sample, wherein the method comprises: (a) providing a sample comprising a ligand, wherein the sample is suspected of comprising a target protein that forms a reversible complex with the ligand; (b) applying the sample to the packed DCC bed of an apparatus of the invention for a time sufficient for adsorption of unbound ligand to the DCC; (c) eluting the sample from the packed DCC bed; (d) optionally filtering the eluted sample through the filter (if present) below the packed DCC bed; and (e) determining the presence of ligand in the eluted sample, wherein the presence of the ligand in the sample indicates that a target protein that binds to the ligand is present in the sample. As discussed above, the “presence” of the ligand in the sample can be determined in comparison with a suitable control (e.g., two-fold over background).

The invention can also be practiced in a competitive binding format in which competition of two different target proteins for binding to a ligand is assessed. In some embodiments, one of the target proteins is detectably labeled and competition for binding to the ligand between the labeled and unlabeled protein is evaluated.

In practicing the foregoing methods, the amount of the ligand in the eluted sample can be determined by any method known in the art, including but not limited to mass spectrometry, immunoassay methods, gel electrophoresis, detection of a detectable label, and the like.

When the ligand is detectably labeled, the method can further comprise detecting the detectably labeled ligand in the eluted sample. Detectable labels are as described above in Section IIIA.

In particular embodiments, a fluorescent displacement assay is used in which unlabeled ligand displaces a fluorescent probe from its cognate binding site on the target protein. Suitable fluorescent probes for use in these methods include but are not limited to dansylamide and dansylsarcosine probes, which bind to subdomains IIA and IIIA of human serum albumin, respectively, and quinaldine red probes, which are specific for the major binding site of AAG. Other suitable probes include fluorescent diazepam analogs, which are specific for subdomain IIA of human serum albumin. These probes are available from commercial sources.

Fluorescent displacement assays can be advantageously used to understand the kinetics/physics of binding between the protein and ligand and/or to provide a rapid method to detect binding between the target protein and ligand.

In particular embodiments of the foregoing methods, the method further comprises pre-incubating the target protein and ligand prior to contacting the sample with the packed DCC bed for a time sufficient for binding. For example, the target protein and ligand can be mixed together and incubated. The “time sufficient for binding” can be any suitable time and in exemplary embodiments can be from about 0.5, 1, 2, 3, 4, 5, 10, 15, 20, 30 or 60 minutes until about 10, 15, 20, 30, 60, 90, 120, 150, 180, 240, 300, 480 or 600 minutes or longer at any suitable temperature (e.g., at room temperature, 37° C., or at 4° C.).

Packed DCC beds are as described above with respect to the inventive apparatus. Further, as described above in Section IIIA with respect to protein binding methods, it may be advantageous to “precondition” the packed DCC bed so as to reduce adsorption of the target protein thereto.

The target protein can be any protein (or portion thereof) of interest including but not limited to plasma proteins, receptors, binding proteins, and enzymes. In particular embodiments, the target protein is a protein found in the circulating blood of a warm-blooded vertebrate (e.g., mammals and avians). For example, the target protein can be a plasma protein, such as serum albumin (e.g., HSA) or AAG. Mammals include but are not limited to humans, non-human primates, dogs, cats, pigs, goats, sheep, cattle, horses, mice, rats and rabbits. Avian subjects include but are not limited to chickens, turkeys, ducks, geese, quail, and birds kept as pets (e.g., parakeets, parrots, macaws, cockatoos, and the like).

The target protein can be isolated or can be present in a mixture of proteins. The target protein can further be a protein found within a biological matrix, such as blood or blood plasma. According to this particular aspect of the invention, providing a sample can comprise contacting a biological matrix (e.g., plasma such as human plasma) comprising a target protein with at least one ligand for a time sufficient for binding of the at least one ligand by the target protein. Further, contacting a biological matrix comprising a target protein with at least one ligand can comprise creating a suspension of the biological matrix comprising a target protein and the at least one ligand. A time sufficient for binding will typically comprise a duration equal to or less than about 15, 30, 45, 60, 90, or 120 minutes (e.g., at room temperature, 37° C. or at 4° C.). In some embodiments of the invention, the target protein is serum albumin (e.g., bovine serum albumin or human serum albumin), α-acid-glycoprotein (AAG), retinoid binding protein, or thyroxin binding protein.

In representative embodiments of the invention, the step of providing a target protein and a ligand comprises contacting a biological matrix (e.g., plasma) comprising the target protein with the ligand for a sufficient time for binding between the target protein and the ligand.

Thus, in particular aspects, the invention provides a method of measuring a target protein in blood plasma (e.g., human blood plasma), wherein the method comprises: (a) providing a sample comprising blood plasma and a ligand, wherein the blood plasma is suspected of comprising a target protein that forms a reversible complex with the ligand; (b) applying the sample to the packed DCC bed of the apparatus of the invention for a time sufficient for adsorption of unbound ligand to the DCC bed; (c) eluting the sample from the packed DCC bed; (d) optionally filtering the eluted sample through the filter (if present) below the packed DCC bed; and (e) determining the amount of ligand in the eluted sample to thereby measure the target protein in blood plasma.

According to other embodiments, the invention provides a method of detecting the presence of a target protein in blood plasma that binds to a ligand, wherein the method comprises: (a) providing a sample comprising blood plasma and a ligand, wherein the blood plasma is suspected of comprising a target protein that forms a reversible complex with the ligand; (b) applying the sample to the packed DCC bed of an apparatus of the invention for a time sufficient for adsorption of unbound ligand to the packed DCC; (c) eluting the sample from the packed bed DCC; (d) optionally filtering the eluted sample through the filter (if present) below the packed DCC bed; and (e) determining the presence of ligand in the eluted sample, wherein the presence of the ligand in the sample indicates that a target protein that binds to the ligand is present in the blood plasma. As discussed above, the “presence” of the ligand in the sample can be determined in comparison with a suitable control (e.g., two-fold over background).

The ligand is generally exogenously added to the sample and can be any molecule known in the art, including but not limited to a protein, a peptide, a saccharide, an oligonucleotide, a lipid, a small chemical molecule, or combinations thereof. Suitable chemical molecules encompass numerous chemical classes, although typically they are organic molecules, preferably small organic compounds having a molecular weight of more than about 100 and less than about 2,500 daltons. Exemplary ligands include ibuprofen, ibuprofen analogs (e.g., naproxen, ketorolac, ketoprofen, etodolac), 5′ 5+ diphenylhydantoin, valproic acid, propranolol, verapamil, diazepam, and chlorpromazine.

In particular embodiments, the ligand is a drug or a candidate drug, e.g., a drug or candidate drug being evaluated for ADMET properties.

The sample is contacted with (e.g., applied to) the packed DCC bed for a time sufficient for adsorption of unbound ligand to the packed DCC bed, which can be any suitable time. In general, however, the methods of the invention are quite rapid as compared with conventional methods. In particular embodiments, the time sufficient for adsorption of unbound ligand to the packed DCC bed is less than or equal to about 120, 90, 60, 45, 30, 20, 15, 10, 8, 6, 5, 4, 3, 2 or even 1 second.

Other aspects of the apparatus, method, ligand, target protein, and the like are as described above in Sections II and IIIA.

IV. Sample Preparation for Complex Matrices.

The apparatus of the invention can also be used in the preparation of biological matrices (e.g., plasma such as human plasma) to reduce the amount of low molecular weight components (e.g., peptides, carbohydrate, lipids including triglycerides, fatty acids, steroids such as cholesterol, and the like, etc.), while leaving the target protein (e.g., a binding protein such as a receptor), preferably in a substantially intact and active form. The low molecular weight components are often ligands that weakly bind to the larger biomolecules (such as proteins) in the sample.

In representative embodiment, a “low molecular weight component” has a molecular weight of less than about 10,000 daltons.

Thus, according to this aspect of the invention, the methods and apparatus are being used in a separation process. Unlike the methods described above, both bound and free ligand are removed via adsorption to the packed DCC bed. The process can be cycled, i.e., the sample can be repeatedly passed through bed to achieve the desired level of separation (i.e., removal of components).

Methods of “stripping” biological matrices, such as plasma, are known in the art. In conventional methods, a relatively large amount of activated charcoal and a sample are mixed and incubated with stirring for two or more hours. Surprisingly, the “stripping” methods of the present invention can be carried out in a matter of minutes or even seconds.

In illustrative embodiments, this aspect of the invention provides a method for preparing a sample by reducing an amount of a low molecular weight component(s) from the sample, wherein the method comprises: (a) providing a sample comprising a biological matrix; (b) applying the sample to the packed DCC bed of an the apparatus of the invention for a time sufficient for adsorption of low molecular weight components to the packed DCC bed; (c) eluting the sample from the packed DCC bed; and (d) optionally filtering the eluted sample through the filter (if present) below the packed DCC bed, to thereby prepare a sample having a reduced amount of a low molecular weight component(s). In embodiments, the eluted sample is not filtered and an apparatus that does not comprise a filter below the DCC bed is used.

In general, those skilled in the art will appreciate that the longer the residence time of the sample in the packed DCC bed (for example, because of reduced flow rate, larger bed volume and/or greater bed depth), the more components that are removed from the biological matrix. With longer residence times, even high binding ligands are removed from the sample due to the on/off rate from their binding partners in the biological matrix. Thus “a time sufficient for adsorption of low molecular weight components” to a packed DCC bed will depend on the bed size and residence times. In particular embodiments, “a time sufficient for adsorption of low molecular weight components” to a packed DCC bed is at least about 2, 5, 10, 15, 20, 30, 60, 120 seconds or more. Suitable ranges includes from about 2 to 60 seconds, 5 to 30 seconds, or 5 to 15 seconds.

According to some embodiments of the invention, at least about 50%, 65%, 75%, 80%, 85%, 90%, 95% or more of a particular component is removed from the sample, or even all or essentially all (e.g., at least 97%, 98% or 99% or more) is removed from the sample.

In a 96-well plate, a typical amount of DCC in the packed bed is from about 7.5 to 15, 20, 25, 30 mg or greater. Sample residence times in the packed bed are typically from about 5, 10 or 15 seconds or greater. For example, the residence time can be from about 5 to 10, 15, 30, 60 or 120 seconds. In one exemplary embodiment, the packed bed is formed from 10 mg of packed DCC in a well of a 96-well plate and the residence time is less than about 5 seconds.

One use of this aspect of the invention is to remove an endogenous component from the biological matrix, so that a defined amount of exogenous component can be added back to the sample. For example, it may be desirable to remove all or essentially all of the estrogens from a sample, so that defined amounts of estrogens can be added back to the stripped sample without interference from the presence of the endogenous estrogens.

The sample preparation methods of the invention further allow for the use of “cleaner” samples with fewer interfering components.

Samples prepared according to this aspect of the invention can further be used in the binding and quantitation assays described herein.

For use in methods of sample preparation by removing low molecular weight components, the apparatus, packed DCC bed, methods of applying the sample and eluting the sample from the matrix are as described above in Sections II, IIIA and IIIB, although in particular embodiments the packed DCC bed is not preconditioned. In other embodiments, the packed DCC bed is preconditioned (as described above).

The invention will now be illustrated with reference to certain examples which are included herein for the purposes of illustration only, and which are not intended to be limiting of the invention.

### EXAMPLE 1

### Evaluation of Dextran-Coated Charcoal (DCC) Device in 96-Well Format

The purpose of this study was to evaluate the scaling down and adaptation of a DCC-based device to a 96-well format.

Experimental:

Preparation of DCC plate. The device used in these studies is identified by the name ACCUPRO™, a propriety mark of Qualyst, Inc. Briefly, prepare a slurry of 0.5 grams of Sigma brand dextran coated charcoal (part# C6197-20G) in 10 mL of 10% w/w dextran (64K-76K) in phosphate buffer saline, pH 7.2 (PBS). Add 50 μL of this slurry (2.5 mg total DCC) to a well in a Millipore HTS DV, 0.65 μm, Hydrophil Durapore® 96-well filter plate (part # MSDVN6510). Apply centrifugal force to move the DCC to the bottom of the plate. Load 250 μL of PBS to the well and layer on top of the PBS a disk of 0.65 μm, Hydrophil Durapore® filter membrane cut to the diameter of the well. Apply centrifugal force to pass the PBS through the DCC bed and press the filter membrane against the DCC bed. Apply gentle pressure against the filter membrane disk to insure that it is touching the DCC. Keep plate covered and at 5° C. until use within 8 hours.

Set-Up of Incubation Study. Prepare 40 mg/mL human serum albumin (HSA) in PBS. Prepare individual samples of 50 μL HSA containing the following drugs at the concentrations indicated: 5,5-Diphenylhydantoin (59.5 μM), Valproic Acid (69.3 μM), Propranolol (racemic) (77 nM), Verapamil (264 nM), and Diazepam (1.0 μM). Sufficient samples are produced such that for each drug at least one sample is analyzed after passage through the ACCUPRO™ device ((+) DCC) and one sample, without exposure to DCC ((−) DCC), is also analyzed. After mixing each sample is allowed to sit at room temperature for 1 hour before DCC extraction.

DCC Extraction Procedure. Place the DCC plate in a 96-well based vacuum manifold; add 100 μL PBS to each well and apply a vacuum at approximately 5″ Hg. Follow this rinse with the addition of 50 μL of 40 mg/mL HSA in PBS and then apply vacuum at 5″ Hg. Wait for 2 minutes after this step. Add to the vacuum manifold a Millipore Multiscreen® Deepwell Solvinert filter plate (part # MDRPNP410). This filter plate has a hydrophobic chemically-resistant hydrophobic polytetrafluoroethylene (PTFE) membrane (with a pore size of 0.45 μm) and a polypropylene prefilter. This filter plate is located directly below the DCC plate and will collect the liquid added to each well of the DCC plate, after application of a vacuum. Add the 50 μL incubation sample to the well in the DCC plate and immediately apply vacuum at 10″ Hg. This step is followed immediately with the addition of 75 μL of PBS with a vacuum application of 10″ Hg.

Protein Precipitation Procedure. To recover the drug from the HSA solution that passed through the DCC plate into the Solvinert plate, 375 μL acetonitrile (with an internal standard [for analysis by mass spectrometry]) is added to the Solvinert filter plate. Individual drug-HSA incubation samples that did not pass through the DCC plate are also added to empty wells in the same Solvinert plate and the acetonitrile/internal standard solution is then added to these non-DCC extracted samples, as well. The Solvinert plate is covered and rotated for approximately 15 minutes. The protein free supernatant from the Solvinert plate is recovered from the filtration plate into a standard deep well collection plate with centrifugation.

Final Sample Preparation. The supernatant is evaporated to dryness, under nitrogen at 45° C., reconstituted in an appropriate sample diluent, mixed, and filtered prior to analysis.

Chemical Analysis. The analysis of (+) DCC and (−) DCC extracted drugs and the internal standard is conducted with liquid chromatography coupled to mass spectrometry with electrospray ionization. For a given sample, the peak areas of the particular drug and its internal standard are measured and a ratio of these two values is determined. The peak area ratio (PAR) of the (+) DCC-extracted sample is divided by its corresponding (−) DCC extracted sample PAR to determine a percent recovery value. This recovery value is not a % HSA bound value.

Results and Discussion:

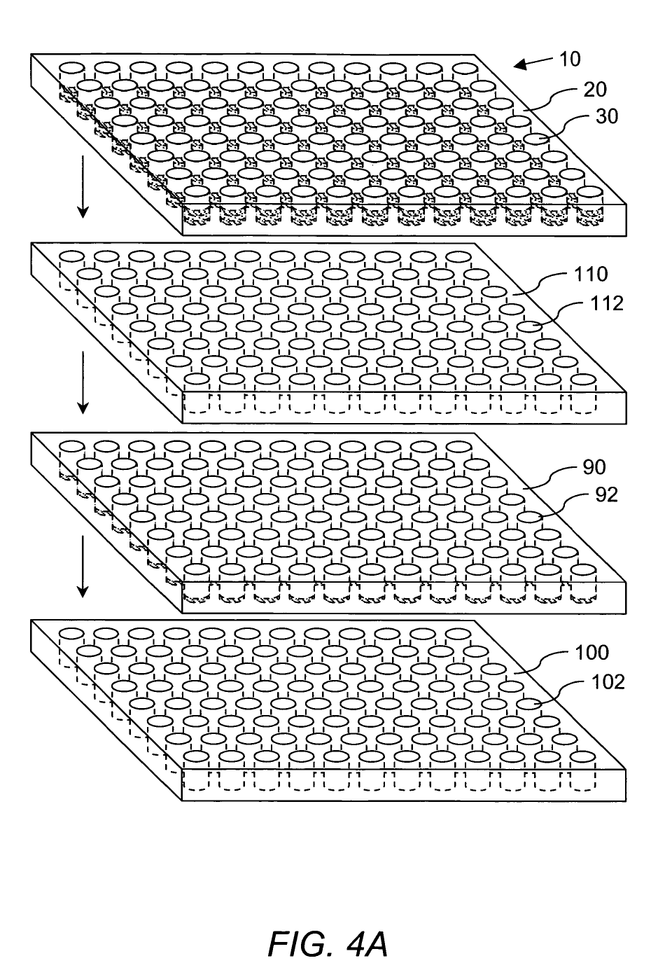

The results of this study are shown in FIG. 5. The x-axis incorporates the HSA unbound fraction values for each drug as reported in International Patent Publication WO 03/015871. The percent recovery values (y-axis) represent an average value (n=3) for all drugs, except valproic acid (n=2). The correlation represented here, for a 96-well format, is comparable to that determined with the DCC device described in International Patent Publication WO 03/015871.

### EXAMPLE 2

### Analysis of α1-Acid Glycoprotein (AAG) in the Presence of HSA

The purpose of this study was to use the 96-well format ACCUPRO™ device described in Example 1 for the analysis of the protein, AAG, in the presence of HSA.

Experimental:

Preparation of DCC plate. The same procedure was utilized as was described in Example 1.

Set-Up of Incubation Study. Under typical human physiological conditions, the concentrations of HSA and AAG in human plasma are approximately 588 μM (40 mg/mL) and 20 μM (0.9 mg/mL), respectively. One stock solution of HSA and AAG was prepared in PBS at these physiological concentrations. A second stock solution was prepared with the same HSA concentration, but with AAG reduced 50% (10 μM). These two stock solutions were diluted 1:3 with PBS, yielding two working solutions of HSA/AAG at 147 μM/5 μM and 147 μM/2.5 μM. In duplicate, a 50 μL aliquot of each working solution was 19 μM in chlorpromazine. Chlorpromazine is reported to be 95-98% plasma bound and has a high affinity to AAG and not HSA. These samples were incubated for 1.5 hours at room temperature, prior to DCC extraction.

DCC Extraction Procedure; Protein Precipitation Procedure; Final Sample Preparation; Chemical Analysis. The same procedures were utilized as were described in Example 1, except that no samples were prepared that did not undergo DCC extraction (no (−) DCC samples), and only the PAR values for chlorpromazine were determined.

Results and Discussion:

The ratio of the calculated PAR values for chlorpromazine in the two incubated working solutions is nearly identical to the ratio of the molar concentration of AAG in the two working solutions. Thus, AAG could be detected proportionally at 1× and 0.5× its typical human physiological concentration while in the presence of a physiologically relevant amount of HSA.

### EXAMPLE 3

### Evaluation of Percent Recovery of Ibuprofen and Four Analogs from Both HSA and Human Plasma Utilizing the 96-Well ACCUPRO™ Device

This experiment was carried out to determine if the 96-well ACCUPRO™ device can discriminate drugs reported as greater than 99% bound in plasma.

Experimental:

Preparation of DCC plate. Same as previously described in Example 1 except the filter on top of the DCC bed is a Millipore HTS, BV 1.2 μM Hydrophil Durapore® Membrane part # MSBVN1210. To prepare DCC plate with a 5 mg bed, add 100 μL of 0.5 g/mL DCC slurry. To prepare a 10 mg DCC bed, add 100 μl of 0.5 g/mL DCC slurry, apply centrifugal force to pack bed, add an additional 100 μL DCC slurry. A (−) DCC control is prepared by loading 250 μL PBS to an empty well followed by a 1.2 μM top filter and applying centrifugal force.

Set-Up of Incubation Study. Prepare 40 mg/mL HSA in PBS. Prepare samples in triplicate for both (+) DCC and (−) DCC extraction of 50 μL HSA or 50 μL human plasma (BioChemed part # 754PRP) containing the following drugs at the concentration indicated: Ibuprofen (48.5 μM), Ketoprofen (1.2 μM), Ketorolac (0.5 μM), Naproxen (217 μM), and Etodolac (45 μM). Allow samples to sit at room temperature for one hour.

DCC Extraction Procedure. As described in Example 1 with the following changes: After initial addition of 100 μL PBS, add 50 μL HSA or 50 μL human plasma, consistent with sample matrix. Add 500 μL acetonitrile (containing internal standard) to Millipore Solvinert filter plate just prior to DCC extraction. Add 50 μL incubated sample to well in the DCC plate (this includes (+) DCC and (−) DCC wells) and immediately apply vacuum at 12″ Hg. This step is followed with addition on 75 μL PBS with vacuum application of 12″ Hg.

Protein Precipitation Procedure. The drug-HSA and drug-human plasma solutions that passed through the DCC plate are collected in a Solvinert plate already containing acetonitrile/internal standard. The Solvinert plate is covered and rotated for approximately 15 minutes. The protein free supernatant is recovered into a standard deep well collection plate with centrifugation.

Final Sample Preparation; Chemical Analysis. As described in Example 1.

Results and Discussion:

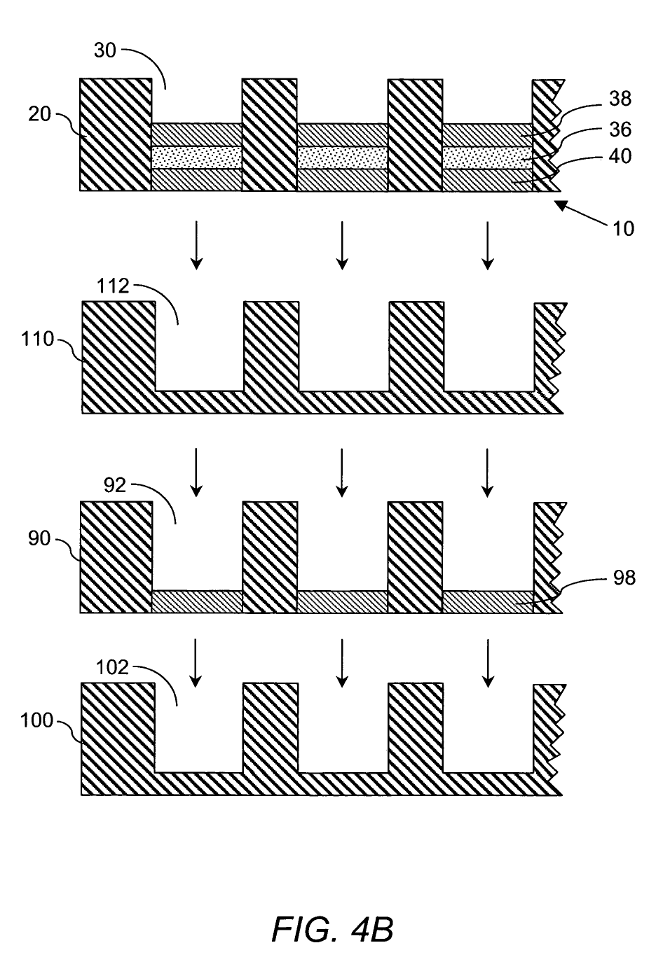

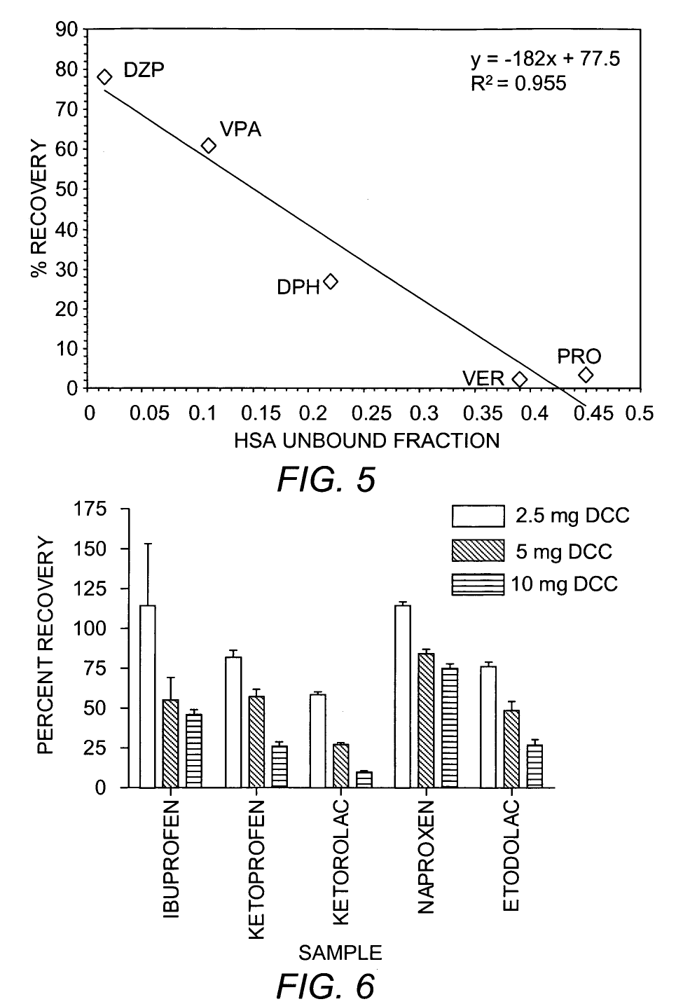

The results of this experiment are shown in FIGS. 6 and 7. These data suggest that the ACCUPRO™ device is capable of discriminating between drugs which are >99% bound in plasma. The ability of the device to resolve these high binders improves with increasing DCC bed size up to 10 mg (largest bed size tested) in both HSA and human plasma, with more pronounced differences in HSA.

### EXAMPLE 4

### Evaluation of Drugs with Consistently High and Low Percent Recovery at the Same Molar Concentration

The purpose of this experiment was to determine the effect of drug concentration on percent recovery from HSA.

Experimental:

Preparation of DCC plate. Prepare a DCC filter plate with 5 mg and 10 mg bed sizes as described in Example 1 and 3.

Set-Up of Incubation Study. Prepare triplicate samples of 50 μL HSA containing Naproxen or Ketorolac at 1 μM. Allow samples to sit at room temperature for one hour.

DCC Extraction Procedure; Protein Precipitation Procedure; Final Sample Preparation; Chemical Analysis: Perform as described in Examples 1 and 3.

Results and Discussion:

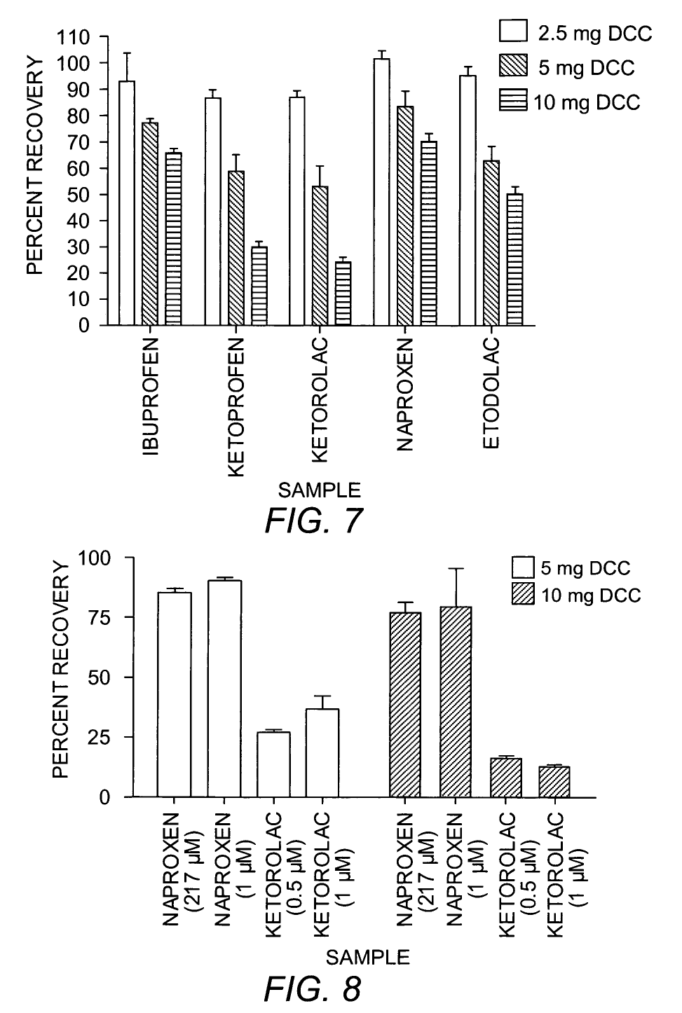

The results are shown in FIG. 8. These data suggest that the drugs tested (Naproxen and Ketorolac, normally run at 217 μM and 0.5 μM respectively), incubated at a selected concentration of 1 μM, have percent recovery values with the ACCUPRO™ device comparable to values obtained above.

### EXAMPLE 5

### Utilization of a Fluorescent Probe in the ACCUPRO™ Device

This study was carried out to evaluate displacement of Dansylsarcosine (DS) as a potential measurement alternative to mass spectrometry for the ACCUPRO™ device. Dansylsarcosine is a fluorescent probe with binding affinity for the same site on HSA as ibuprofen and ibuprofen analogs.

Experimental:

Preparation of DCC plate. Prepare a DCC filter plate with a 10 mg bed size as described in Example 1 and 3. (−) DCC wells were not prepared because a percent recovery value is not calculated.

Set-Up of Incubation Study. Prepare triplicate samples of 50 μL HSA containing Naproxen or Ketorolac at 1 μM and 1 μM Dansylsarcosine (DS). Allow samples to sit at room temperature for one hour.

DCC Extraction Procedure; Protein Precipitation Procedure; Final Sample Preparation. Performed as described in Example 1 and 3.

Chemical Analysis. The analysis of DS and the internal standard is conducted with liquid chromatography coupled to mass spectrometry with electrospray ionization. For a given sample, the peak area of the DS and the internal standard are measured and a ratio (PAR) of these two values is determined.

Results and Discussion:

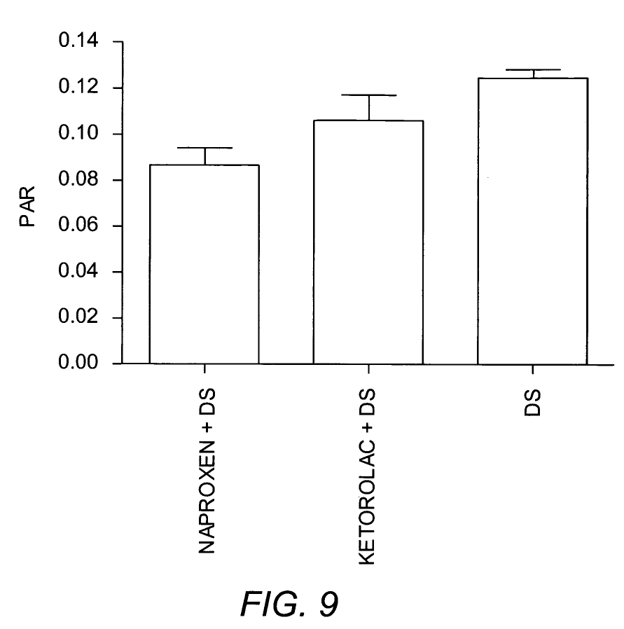

The results for this study are shown in FIG. 9. A drug with a higher percent recovery (Naproxen), suggesting a higher percent bound in HSA should displace the competing fluorescent probe (DS) to a greater extent than a drug that is bound to a lesser extent. When the DS is displaced, it is captured in the DCC bed and will be present at a lower level in the final sample preparation, as observed in Naproxen+DS sample. A drug with a lower percent recovery (Ketorolac) will displace less of the DS probe. With more DS remaining bound to HSA, a higher PAR is observed. When no drug is present, DS is maximally bound by HSA, yielding the highest PAR.

The foregoing is illustrative of the present invention, and is not to be construed as limiting thereof. The invention is defined by the following claims, with equivalents of the claims to be included therein.

That which is claimed:

1. An apparatus comprising a multiwell plate, each well of the multiwell plate comprising:
(a) a bottom portion having an opening formed therein;
(b) a packed dextran-coated activated charcoal (DCC) bed;
(c) a filter membrane on top of the packed DCC bed which covers the exposed upper surface thereof; and
(d) a filter membrane below the packed DCC bed, wherein the filter membrane covers the opening.

2. The apparatus of claim 1, wherein the DCC comprises dextrans having an average molecular weight of 50 kDa to 150 kDa.

3. The apparatus of claim 1, wherein the DCC comprises a fractional weight of 10% to 80% dextran.

4. The apparatus of claim 1, wherein the multiwell plate is a 96-well filtration plate.

5. The apparatus of claim 4, wherein the packed DCC bed comprises from 0.5 mg to 15 mg of DCC.

6. The apparatus of claim 5, wherein the packed DCC bed comprises from 7.5 mg to 15 mg of DCC.

7. The apparatus of claim 5, wherein the packed DCC bed comprises from 1 mg to 3 mg of DCC.

8. The apparatus of claim 1, wherein the packed DCC bed is from 0.25 to 2 mm in thickness.

9. The apparatus of claim 1, wherein the multiwell plate comprises a drain that leads to a second multiwell plate.

10. The apparatus of claim 9, wherein the second multiwell plate is a filtration plate.

11. The apparatus of claim 10, wherein the second multiwell plate comprises a drain that leads to a third multiwell plate.

12. A kit comprising the apparatus of claim 1 packaged together with written instructions for methods for determining protein-ligand binding, protein detection and/or quantitation, sample preparation and/or diagnostic methods, and optionally additional reagents or apparatus for carrying out such methods.

13. A method of evaluating binding of a ligand to a target protein, wherein the method comprises:
(a) providing a sample comprising a target protein and a ligand, wherein the target protein and the ligand are suspected to form a reversibly bound complex;
(b) applying the sample to the packed DCC bed of the apparatus of claim 1 for a time sufficient for adsorption of unbound ligand to the DCC;
(c) eluting the sample from the packed DCC bed;
(d) filtering the eluted sample through the filter membrane below the packed DCC bed; and
(e) determining an amount of ligand in the eluted sample to thereby evaluate binding of the ligand to the target protein.

14. The method of claim 13, further comprising preconditioning the packed DCC bed prior to the step of applying the sample.

15. The method of claim 13, wherein the providing step comprises contacting the target protein and the ligand for a time sufficient for binding between the target protein and the ligand.

16. The method of claim 13, wherein the target protein is a protein found in the circulating blood of a warm-blooded vertebrate, and is optionally albumin, α-acid-glycoprotein, retinoid binding protein, or thyroxin binding protein.

17. The method of claim 13, wherein the determining step comprises determining a fractional amount of the ligand that is bound to the target protein.

18. The method of claim 13, wherein the determining step comprises determining a percent recovery of ligand, where percent recovery=amount of ligand that is recovered from the apparatus/amount of ligand that is recovered in a control sample that has not been applied to the packed DCC bed.

19. The method of claim 13, wherein the percent ligand bound to the target protein is at least 97% or higher.

20. The method of claim 19, wherein the percent ligand bound to the target protein is at least 99% or higher.

21. The method of claim 13, wherein the ligand comprises a peptide, an oligonucleotide, a small chemical molecule, or a combination thereof.

22. The method of claim 13, wherein the ligand is a xenobiotic, a candidate drug, or a new chemical entity.

23. The method of claim 13, wherein the ligand is detectably labeled and wherein the step of determining the amount of ligand in the eluted sample comprises detecting the detectably labeled ligand.

24. The method of claim 23, wherein the detectable label is a fluorescent label, a luminescent label, an epitope label, a calorimetric label, or a radiolabel.

25. The method of claim 13, wherein the step of determining the amount of ligand in the eluted sample comprises mass spectrometry.

26. The method of claim 13, wherein the time sufficient for adsorption of unbound ligand to the packed DCC bed is 5 seconds or less.

27. The method of claim 13, wherein the eluted sample is collected in a second multiwell plate.

28. The method of claim 27, wherein the determining step comprises denaturing the target protein in the eluted sample collected in the second multiwell plate.

29. The method of claim 28, wherein the free ligand is separated from the denatured target protein by filtration.

30. The method of claim 29, wherein the second multiwell plate is a filtration plate and the free ligand is separated from the denatured target protein by filtration in the second multiwell plate.

31. The method of claim 29, wherein the second multiwell plate is a transfer plate and all or a portion of the sample comprising the free ligand and denatured target protein is transferred from the transfer plate to a multiwell filtration plate and the free ligand is separated from the denatured target protein by filtration in the multiwell filtration plate.

32. The method of claim 13, wherein the sample comprises a mixture of proteins comprising the target protein.

33. The method of claim 32, wherein the sample comprises a biological matrix comprising the target protein.

34. The method of claim 33, wherein the biological matrix comprises blood plasma.

35. The method of claim 34, wherein the target protein comprises serum albumin, α-acid-glycoprotein, retinoid binding protein, or thyroxin binding protein.

36. The method of claim 13, wherein the method comprises:
(a) providing a sample comprising a target protein and a first ligand, wherein the first ligand forms a reversible complex with the target protein;
(b) contacting the sample with a candidate second ligand for a time sufficient for displacement of the first ligand from the complex by the candidate second ligand;
(c) contacting the sample of (b) with the packed DCC bed for a time sufficient for adsorption of unbound first ligand to the DCC;
(d) eluting the sample from the packed DCC bed;
(e) filtering the eluted sample through the filter membrane below the packed DCC bed; and
(f) determining an amount of first ligand in the eluted sample to thereby evaluate binding of the candidate second ligand to the target protein.

37. The method of claim 36, wherein the first ligand is detectably labeled and wherein the step of determining the amount of the first ligand in the eluted sample comprises detecting the detectably labeled first ligand.

38. The method of claim 37, wherein the step of determining the amount of ligand in the eluted sample comprises a fluorescent displacement assay.

39. The method of claim 36, wherein the step of determining the amount of first ligand in the eluted sample comprises mass spectrometry.

40. The method of claim 36, wherein the determining step comprises determining a fractional amount of the first ligand that is bound to the target protein.

41. The method of claim 36, wherein the first ligand and the candidate second ligand each comprise a peptide, an oligonucleotide, a small chemical molecule, or a combination thereof.

42. The method of claim 36, wherein the target protein is a protein found in the circulating blood of a warm-blooded vertebrate, and is optionally serum albumin, α-acid-glycoprotein, retinoid binding protein, or thyroxin binding protein.

43. The method of claim 36, wherein the first ligand comprises a ligand that binds a specific binding site on the target protein.

44. The method of claim 43, wherein the first ligand comprises a ligand that binds to a protein found in the circulating blood of a warm-blooded vertebrate, wherein the protein is optionally serum albumin or α-acid-glycoprotein.

45. The method of claim 43, wherein the first ligand comprises a ligand that binds site I, site II, or site III of human serum albumin.

46. The method of claim 45, wherein the first ligand comprises a site I-binding ligand selected from the group consisting of a coumarin and a pyrazolidine.

47. The method of claim 46, wherein the first ligand comprises a ligand selected from the group consisting of valproate, diphenylhydantoin, or salicylate.

48. The method of claim 45, wherein the first ligand comprises a site II-binding ligand selected from the group consisting of a benzodiazepine, an arylpropionate, and L-tryptophan.

49. The method of claim 48, wherein the first ligand comprises diazepam.

50. The method of claim 45, wherein the first ligand comprises the site III-binding ligand digitoxin.

51. The method of claim 36, wherein the first ligand is ibuprofen or an ibuprofen analog.

52. The method of claim 36, wherein the sample comprises a mixture of proteins comprising the target protein.

53. The method of claim 52, wherein the sample comprises a biological matrix comprising the target protein.

54. The method of claim 53, wherein the biological matrix comprises blood plasma.

55. The method of claim 54, wherein the target protein comprises serum albumin, α-acid-glycoprotein, retinoid binding protein, or thyroxin binding protein.

56. A method for evaluating the susceptibility of a candidate drug to binding a target protein, wherein the method comprises:
(a) providing a sample comprising a target protein and a ligand, wherein the ligand forms a reversible complex with the target protein;
(b) contacting the sample with a candidate drug for a time sufficient for displacement of the ligand from the complex by the candidate drug;
(c) applying the sample of (b) to the packed DCC bed of the apparatus of claim 1 for a time sufficient for adsorption of unbound ligand to the packed DCC bed;
(d) eluting the sample from the packed DCC bed;
(e) filtering the eluted sample through the filter membrane below the packed DCC bed; and
(f) determining an amount of ligand in the eluted sample to thereby evaluate the susceptibility of the candidate drug to binding the target protein.

57. A method for evaluating drug-drug interactions, wherein the method comprises:
(a) providing a sample comprising a target protein and a ligand; wherein the ligand forms a reversible complex with the target protein;
(b) contacting the sample with a first candidate drug in the presence of a second candidate drug for a time sufficient for displacement of the ligand from the complex by the first candidate drug;
(c) applying the sample of (b) to the packed DCC bed of the apparatus of claim 1 for a time sufficient for adsorption of unbound ligand to the packed DCC bed;
(d) eluting the sample from the packed DCC bed;
(e) filtering the eluted sample through the filter membrane below the packed DCC bed;
(f) repeating steps (a) to (e) in the absence of the candidate second drug; and
(g) determining an amount of ligand in the eluted sample in the presence of the second candidate drug and comparing with an amount of ligand in the absence of the candidate drug to thereby evaluate interactions between the first and second candidate drugs.

58. A method of measuring a target protein in a sample, wherein the method comprises:
(a) providing a sample comprising a ligand, wherein the sample is suspected of comprising a target protein that forms a reversible complex with the ligand;
(b) applying the sample to the packed DCC bed of the apparatus of claim 1 for a time sufficient for adsorption of unbound target ligand to the DCC;
(c) eluting the sample from the packed DCC bed;
(d) filtering the eluted sample through the filter membrane below the packed DCC bed; and
(e) determining an amount of ligand in the eluted sample to thereby measure the target protein in the sample.

59. The method of claim 58, wherein the method is a quantitative method and comprises determining the amount of the target protein in the sample.

60. The method of claim 58, wherein the method is a qualitative method and comprises determining the presence or absence of the target protein in the sample.

61. The method of claim 58, wherein the method is a semi-quantitative method and comprises determining the presence or absence of the target protein in the sample above a threshold amount.

62. The method of claim 58, wherein the method is practiced to measure a circulating protein that is a marker protein associated with a disease state.

63. The method of claim 62, wherein the circulating protein is α-acid-glycoprotein.

64. The method of claim 62, wherein the circulating protein is selected from the group consisting of: alpha-feto protein, prostate specific antigen, C-reactive protein, alanine aminotransferase, an autoantibody, an antigen from an infectious agent, a cancer antigen, and a protein associated with an inborn error of metabolism.

65. The method of claim 58, further comprising preconditioning the packed DCC bed prior to the step of applying the sample.

66. The method of claim 58, wherein the providing step comprises contacting the target protein and the ligand for a time sufficient for binding between the target protein and the ligand.

67. The method of claim 58, wherein the target protein is a protein found in the circulating blood of a warm-blooded vertebrate, and is optionally serum albumin, α-acid-glycoprotein, retinoid binding protein, or thyroxin binding protein.

68. The method of claim 58, wherein the determining step comprises determining a fractional amount of the ligand that is bound to the target protein.

69. The method of claim 58, wherein the ligand comprises a peptide, an oligonucleotide, a small chemical molecule, or a combination thereof.

70. The method of claim 58, wherein the ligand is a xenobiotic, a candidate drug, or a new chemical entity.

71. The method of claim 58, wherein the ligand is detectably labeled and wherein the step of determining the amount of ligand in the eluted sample comprises detecting the detectably labeled ligand.

72. The method of claim 71, wherein the detectable label is a fluorescent label, a luminescent label, an epitope label, a calorimetric label, or a radiolabel.

73. The method of claim 72, wherein the step of determining the amount of ligand in the eluted sample comprises a fluorescent displacement assay.

74. The method of claim 58, wherein the step of determining the amount of ligand in the eluted sample comprises mass spectrometry.

75. The method of claim 58, wherein the time sufficient for adsorption of unbound ligand to the packed DCC bed is 5 seconds or less.

76. The method of claim 58, wherein the eluted sample is collected in a second multiwell plate.

77. The method of claim 76, wherein the determining step comprises denaturing the target protein in the eluted sample collected in the second multiwell plate.

78. The method of claim 76, wherein the free ligand is separated from the denatured target protein by filtration.

79. The method of claim 78, wherein the second multiwell plate is a filtration plate and the free ligand is separated from the denatured target protein by filtration in the second multiwell plate.

80. The method of claim 78, wherein the second multiwell plate is a transfer plate and all or a portion of the sample comprising the free ligand and denatured target protein is transferred from the transfer plate to a multiwell filtration plate and the free ligand is separated from the denatured target protein by filtration in the multiwell filtration plate.

81. The method of claim 58, wherein the sample comprises a mixture of proteins comprising the target protein.

82. The method of claim 81, wherein the sample comprises a biological matrix comprising the target protein.

83. The method of claim 82, wherein the biological matrix comprises blood plasma.

84. The method of claim 83, wherein the target protein comprises serum albumin, α-acid-glycoprotein, retinoid binding protein, or thyroxin binding protein.

85. A method of detecting the presence or absence of a target protein in a sample, wherein the method comprises:
(a) providing a sample comprising a ligand, wherein the sample is suspected of comprising a target protein that forms a reversible complex with the ligand;
(b) applying the sample to the packed DCC bed of the apparatus of claim 1 for a time sufficient for adsorption of unbound ligand to the DCC;
(c) eluting the sample from the packed DCC bed;
(d) filtering the eluted sample through the filter membrane below the packed DCC bed; and
(e) determining the presence of the ligand in the eluted sample, wherein the presence of the ligand in the eluted sample indicates that a target protein that binds to the ligand is present in the sample.

86. The method of claim 85, wherein the method is carried out to identify a protein that binds to the ligand.

87. The method of claim 86, wherein the method is carried out to screen plasma fractions.

88. A method for preparing a sample by reducing an amount of low molecular weight components in the sample, wherein the method comprises:
(a) providing a sample comprising a biological matrix;
(b) applying the sample to the packed DCC bed of the apparatus of claim 1 for a time sufficient for adsorption of low molecular weight components to the packed DCC bed;
(c) eluting the sample from the packed DCC bed; and
(d) filtering the eluted sample through the filter membrane below the packed DCC bed to thereby prepare a sample having a reduced amount of low molecular weight components.

89. The method of claim 88, further comprising the step:
(e) using the sample having the reduced amount of low molecular weight components to evaluate binding between a protein and a ligand.

90. The method of claim 88, further comprising the step:
(e) using the sample having the reduced amount of low molecular weight components to measure a protein in the sample.

91. The method of claim 88, wherein the biological matrix comprises blood plasma.

92. The method of claim 88, wherein the low molecular weight components comprise a lipid.

93. The method of claim 88, wherein the low molecular weight components comprises a peptide.

94. The method of claim 88, wherein the low molecular weight components comprise a carbohydrate.

95. The method of claim 88, wherein the multiwell plate is a 96-well plate and the packed DCC bed comprises from 0.5 mg to 15 mg of DCC.

96. The method of claim 95, wherein the multiwell plate is a 96-well plate and the DCC bed comprises from 7.5 mg to 15 mg of DCC.
